(19) United States
(12) Patent Application Publication
(10) Pub. No.: US 2013/0081479 A1
(43) Pub. Date: Apr. 4, 2013

(54) SENSOR SYSTEM TO DETECT PERSENCE OF A PERSON ON AN OBJECT AND MONITORING SYSTEM COMPRISING A SENSOR SYSTEM TO DETECT PERSENCE OF A PERSON

(71) Applicants: CRAIG MILLER, PITTSBURGH, PA (US); MICHAEL STURDEVANT, CONCORD TOWNSHIP, OH (US); WILLIAM KAIGLER, WEXFORD, PA (US)

(72) Inventors: CRAIG MILLER, PITTSBURGH, PA (US); MICHAEL STURDEVANT, CONCORD TOWNSHIP, OH (US); WILLIAM KAIGLER, WEXFORD, PA (US)

(21) Appl. No.: 13/631,971

(22) Filed: Sep. 29, 2012

Related U.S. Application Data
(60) Provisional application No. 61/541,537, filed on Sep. 30, 2011.
(60) Provisional application No. 61/662,752, filed on Jun. 21, 2012.

(51) Int. Cl. G01L 1/02; A61B 5/024; A61B 5/08

(52) U.S. Cl. 73/862.53; 73/862.581; 600/528; 600/500

(57) ABSTRACT

A presence sensor system includes at least one resilient extending member defining an enclosed sensing volume. The sensing volume includes a fluid therein. A pressure within the sensing volume changes upon application of force to the extending member. The presence sensor system further includes a pressure sensor in fluid connection with the sensing volume, a processor system in communicative connection with the pressure sensor and a communication system in communicative connection with the processor system. In a number of embodiments, the presence sensor system is adapted to determine a pressure threshold associated with onset of presence after being placed in use.

### CROSS-REFERENCE TO RELATED APPLICATIONS

This application claims benefit of U.S. Provisional Patent Application Ser. No. 61/541,537, filed Sep. 30, 2011, U.S. Provisional Patent Application Ser. No. 61/662,752, filed Jun. 21, 2012, the disclosures of which are incorporated herein by reference.

### BACKGROUND

The following information is provided to assist the reader to understand the technologies disclosed below and the environment in which such technologies will typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

A number of systems are available to monitor the wellbeing of a person. For example, currently available personal emergency response systems (PERS) provide a wearable communicator actuatable by the user in the case of an emergency. Various clinical monitoring systems can, for example, be used to monitor physiological parameters, such as blood pressure, blood glucose levels, weight, etc. A number of home or office remote monitoring systems are based upon security technology. Many currently available remote monitoring systems and/or methods for monitoring the wellbeing of a person are expensive, difficult to implement, and usually are reactive to changes in the person's condition. As a result, remote caregivers are typically alerted of a problem with the person only in the event of an acute attack or when the person initiates an alert, typically by pressing a button. Moreover, a number of sensors available for use in such monitoring system are not well-suited for the purpose.

### SUMMARY

In one aspect, a presence sensor system includes at least one resilient extending member defining an enclosed sensing volume. The sensing volume includes a fluid therein. The pressure within the sensing volume changes (for example, via a change in volume/compression of the sensing volume) upon application of force to the extending member. The presence sensor system further includes a pressure sensor in fluid connection with the sensing volume, a processor system in communicative connection with the pressure sensor and a communication system in communicative connection with the processor system. In a number of embodiments, the presence sensor system is adapted to determine a pressure threshold associated with onset of presence after being placed in use. The system may also be adapted to change the pressure threshold over time on the basis of measured pressures. For example, the pressure threshold may be determined on the basis of at least one of minimum pressures measured over time and maximum pressures measured over time. In a number of embodiments, the pressure threshold is determined on the basis of at least one of an average minimum pressure determined over time and an average maximum pressure determined over time. In a number of embodiments, the sensor system determines a time associated with the onset of presence and a variable associated with duration of presence. The sensor system may, for example, further include a flow sensor.

The sensing volume may, for example, be filled with air. In a number of embodiments, the sensing volume is filled with air at approximately ambient pressure (for example, as measured when no force is being applied to the resilient extending member).

The sensor system may, for example, be adapted to measure at least one variable associated with the wellbeing of the person after the onset of presence. The pressure sensor may, for example, measure variation in pressure after onset of presence is determined. The measurement of variations in pressure may, for example, be used to determine if the onset of presence is associated with a person (as, for example, opposed to an inanimate object). Variations in pressure may, for example, be associated with at least one of movement or variation in a physiological parameter. The physiological parameter may, for example, be at least one of respiration or pulse.

In a number of embodiments a single resilient extending member is used in the sensor system. The resilient extending member may, for example, be a tube. In a number of embodiments, the tube is filed with air at approximately ambient pressure.

In a number of embodiments, the output of the pressure sensor is connected to an analog to digital converter in a ratiometric configuration and the analog to digital converter is connected to the processor.

In a number of embodiments, pressure averages over defined period of times are determined from measured pressures and a difference between the pressure averages for a subsequent defined period of time and a previous defined period of time are determined. Defined periods of time that are adjacent in time may, for example, be separated by a defined separation period. For example, in one embodiment, 15-second averages (for example, pressure measurement made every second and averaged over a 15-second period of time) were determined and adjacent 15-second averages were separated by a separation period of 60 seconds. In a number of embodiments, the difference between the pressure averages for a subsequent defined period of time and the previous defined period of time is compared to the pressure threshold.

In a number of embodiments, the sensor system further includes an extending support member adjacent to the at least one resilient extending member. The extending support member is more rigid that the at least one resilient extending member. The extending support member may, for example, be adjacent to a side of the at least one resilient extending member generally opposite a side to which force is applied in the case of presence of a load. The extending support member may, for example, be attached to the at least one resilient extending member.

In another aspect, a system for monitoring wellness of a person includes a local system in the vicinity of the person including a plurality of sensor systems. Each of the plurality of sensor systems is adapted to monitor changes in state of at least one monitored system caused by activity or lack of activity of the person. At least one of the plurality of sensor systems is a presence sensor system as described above. The system may also include a local data communication device in communicative connection with each of the plurality of sensor system to receive data from each of the plurality of sensor systems.

In a further aspect, a system for monitoring wellness of a person includes a presence sensor system as described above.

In still a further aspect, a method of monitoring for presence of a person includes placing at least one resilient extending member defining an enclosed sensing volume in operative connection with an item upon which presence is to be determined. The sensing volume includes a fluid therein. The pressure within the sensing volume changes upon application of force to the extending member (resulting in pressure changes). The method further includes measuring pressure via a pressure sensor in fluid connection with the sensing volume, communicating a signal from the pressure sensor to a processor system in communicative connection with the pressure sensor; and providing a communication system in communicative connection with the processor system.

In a number of embodiments, the method is adaptive. The method may, for example, further include determining a pressure threshold associated with onset of presence after placing the resilient extending member in operative connection with the item. The pressure threshold may, for example, be changed over time on the basis of measured pressures. In a number of embodiments, the pressure threshold is determined/changed on the basis of at least one of minimum pressures measured over time and at least one maximum pressure measured over time. The pressure threshold may, for example, be determined/changed on the basis of at least one of an average minimum pressure determined over time and an average maximum pressure determined over time.

The method may further include determining a time associated with the onset of presence and a variable associated with duration of presence. The method may also further include measuring flow.

In a number of embodiments, the sensing volume is filled with air. The air may, for example, be filled with air at approximately ambient pressure.

In a number of embodiments, the method further includes measuring variation in pressure after onset of presence is determined (and is continuing). Measurement of variations in pressure may, for example, be used to determine if the onset of presence is associated with a person. Variations in pressure after onset of presence may, for example, be compared with variations in minimum pressure. The variations in pressure may, for example, be associated with at least one of movement of variation in a physiological parameter. The physiological parameter may, for example, be at least one of respiration or pulse.

In a number of embodiments, a single resilient extending member is placed in operative connection with the item. The resilient extending member may, for example, be a tube. In a number of embodiments, the tube is filled with air at approximately ambient pressure.

The present devices, systems and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a schematic representation an embodiment of a system for collecting data from a plurality of devices for remote wellness monitoring.

FIG. 1B illustrates another schematic representation of the system of FIG. 1A.

FIG. 1C illustrates a another schematic representation of the system of FIG. 1A.

FIG. 2A illustrates a side view an embodiment of an energy sensor system or energy sensor for connection to an electrical outlet and to an electrically powered device or system to be monitored, wherein the energy sensor system is removed from connection with the electrical outlet.

FIG. 2B illustrates the energy sensor system of FIG. 2A in connection with the electrical outlet and a plug of a device to be monitored in alignment for electrical connection to an outlet of the energy sensor system.

FIG. 2C illustrates a schematic diagram of the components of the energy sensor system of FIG. 2A.

FIG. 2D illustrates a circuit diagram of the energy sensor system of FIG. 2A.

FIG. 2E illustrates a flowchart for the operation of an embodiment of an energy sensor system such as the energy sensor system of FIG. 2A.

FIG. 3A illustrates a schematic diagram of a sensor system to detect the presence of a person on, for example, a bed, chair, sofa or other similar item or article of furniture.

FIG. 3B illustrates an embodiment of a sensor system including one of more sensor volumes enclosed by one or more resilient extending members such as tubes, pads etc. in operative connection with a bed.

FIG. 3C illustrates an enlarged side view of one of the extending members of FIG. 3B in fluid connection with a pressure transducer via an intermediate conduit and a cross-sectional view of one of the extending members.

FIG. 3D illustrates a side view of a sensor volume of a sensor system of FIG. 3A positioned between a mattress and a box spring of a bed.

FIG. 3E illustrates an embodiment of a circuit diagram for an embodiment of a sensor system hereof.

FIG. 3F illustrates an embodiment of a flowchart for a methodology for detection of presence of a person in a bed.

FIG. 3G illustrates an embodiment of a flowchart for a methodology for detection of movement of a person in a bed.

FIG. 3H illustrates results of the use of a system hereof and the application of an algorithm or methodology hereof to a bed wherein a threshold is adaptively determined to determine “on” and “off” states.

FIG. 3I illustrates further results of the use of a system hereof and the application of an algorithm or methodology hereof to a bed wherein a threshold is adaptively determined to determine “on” and “off” states.

FIG. 4A illustrates an embodiment of a screen for login and for device rule settings.

FIG. 4B illustrates an embodiment of a screen summarizing set rules for alerts and an embodiment of a screen summarizing resident information.

FIG. 4C illustrates an embodiment of a screen summarizing caregiver information.

FIG. 4D illustrates an embodiment of a screen setting forth an activity summary derived from state-based sensor data.

FIG. 4E illustrates an embodiment of a screen setting forth entertainment activity derived from state-based sensor data.

FIG. 4F illustrates an embodiment of a screen setting forth activity derived from state-based kitchen device sensor data.

FIG. 4G illustrates an embodiment of a screen setting forth sleep activity derived from state-based sensor data.

FIG. 4H illustrates an embodiment of a screen setting forth water use derived from state-based sensor data.

FIG. 5 illustrates a flowchart for an embodiment of methodology for the uploading of data to the remote system, the determination of associated or relevant rules, and the application of such rule to determine whether an alert should be generated.

FIG. 6 illustrates a flowchart for an embodiment of methodology for alerting one or more caregivers via one or more communication devices or systems and including an optional attempt to confirm a monitored person is OK via an attempt to communicate with or contact the monitored person.

### DETAILED DESCRIPTION

As used herein and in the appended claims, the singular forms “a,” “an”, and “the” include plural references unless the content clearly dictates otherwise. Thus, for example, reference to “a sensor” includes a plurality of such sensors and equivalents thereof known to those skilled in the art, and so forth, and reference to “the sensor” is a reference to one or more such sensors and equivalents thereof known to those skilled in the art, and so forth.

In a number or representative embodiments, a remote wellness monitoring system monitors basic day-to-day activities or lack of activity of person 5, such as sleeping behavior, television usage, eating habits, water consumption, etc. An example of such a system is described in U.S. Patent Application Publication No. 2012/0056746, the disclosure of which is incorporated herein by reference. The system provides real time monitoring of parameters indicative of the overall wellbeing of the resident and provides timely alerts designed, for example, to help prevent an acute episode. The system may, for example, be used in conjunction with a personal emergency response system (PERS) or as a standalone system, to provide relatively comprehensive remote monitoring for a remote caregiver at a price and ease of installation that is currently not available.

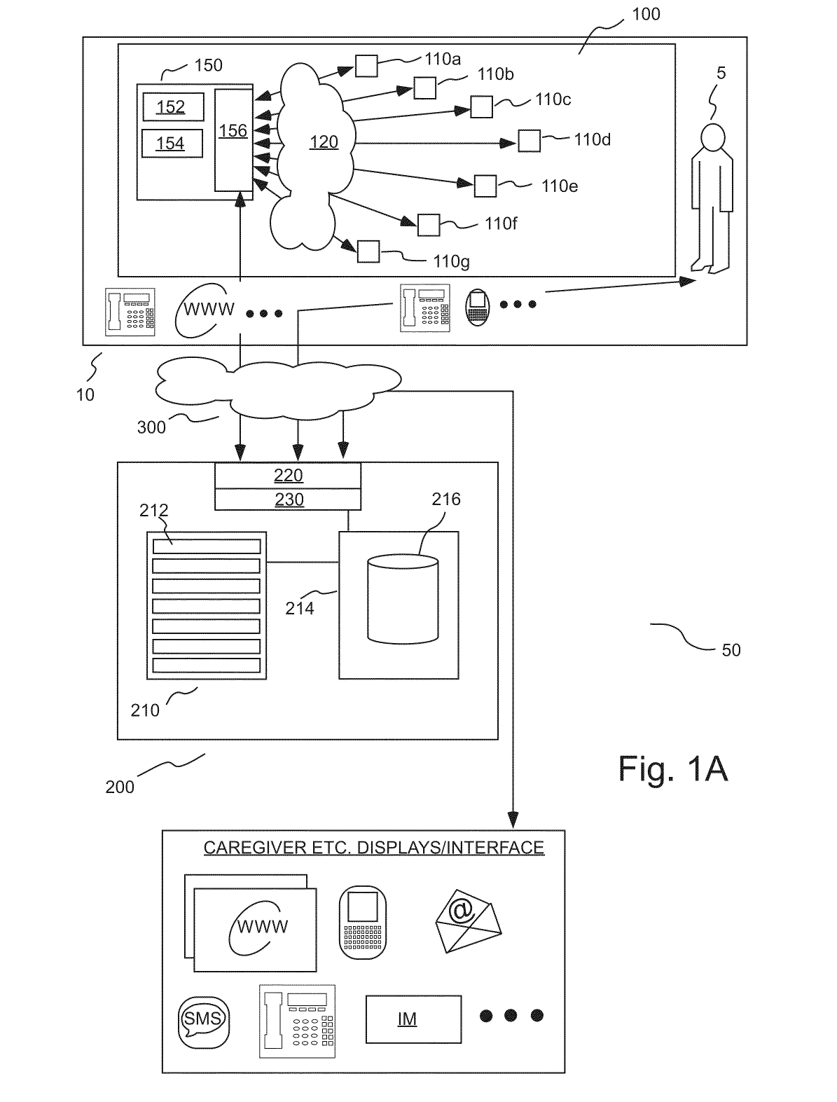

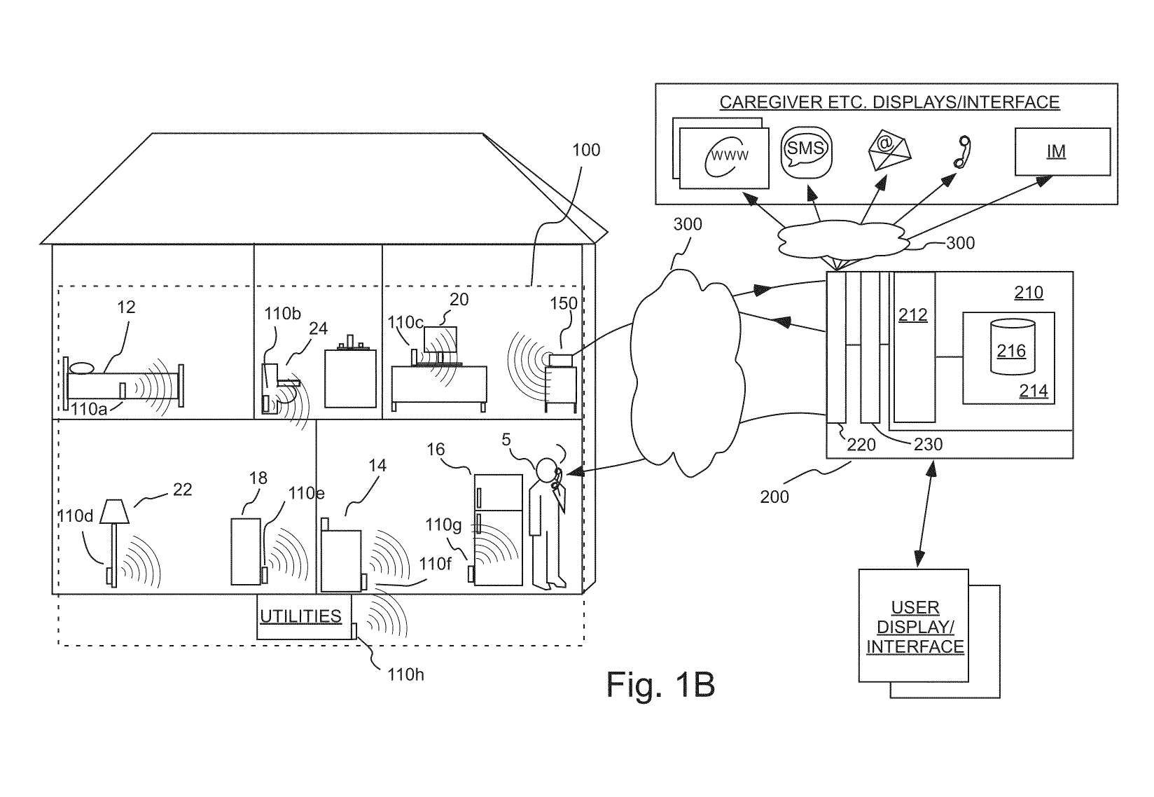

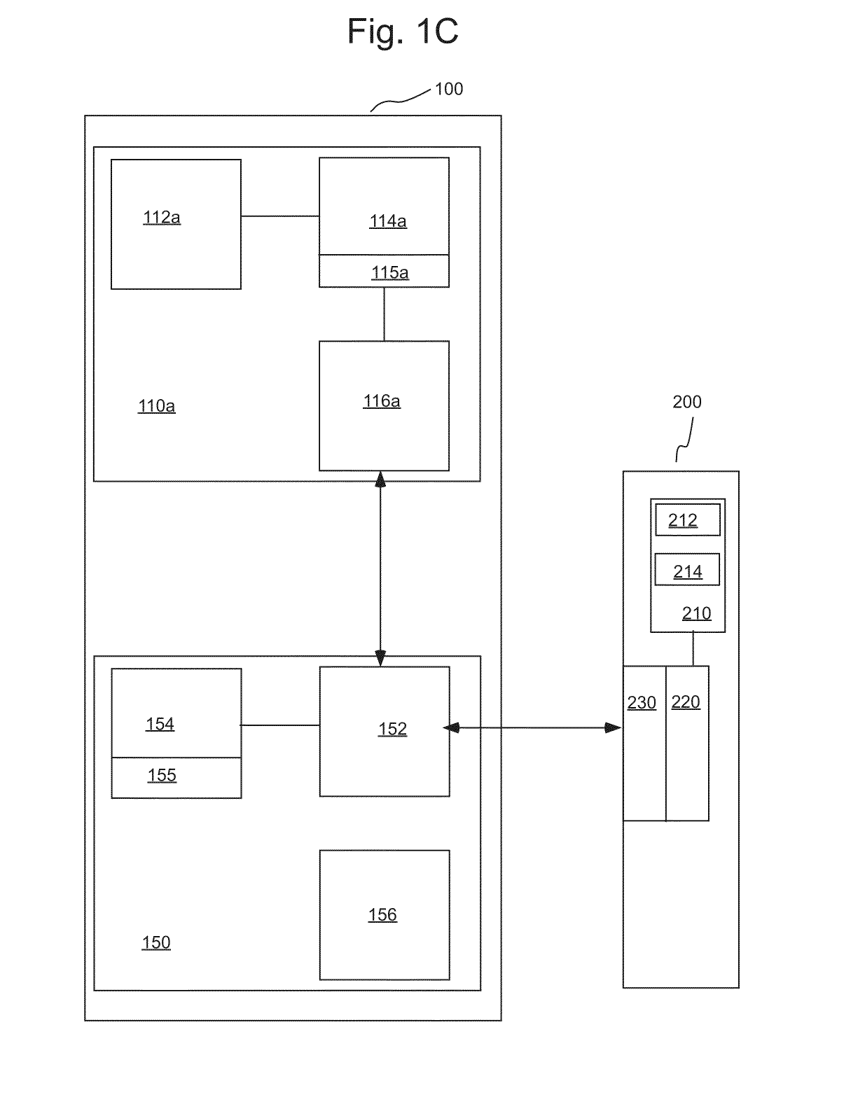

As described further below, while the monitoring of various devices and system in the vicinity of person 5 via a local system 100 (see FIGS. 1A through 1C) is real-time, the transmission of the collected data to a remote system 200, and ultimately to a caregiver (for example, a relative, friend, professional caregiver etc.), may be performed in a discontinuous or batch manner. For example, data of information of and/or a summary of the activity of person 5 for a given period (for example, a prior period of time of 24 hours) can be transmitted by local system 100 to remote system 200 for processing and/or analysis by remote system 200. Remote system 200 can received data from many local systems 100 regarding many different monitored persons 5. Local system 100 may, however, include a processing system including one or more processors programmed or adapted to determine if an emergency or exception event has occurred (based upon data from monitored devices and/or systems) which requires an expedited or unscheduled (for example, immediate) transmission or upload of data or information to remote system 200. Unscheduled uploads resulting from a determined emergency or exception event are sometimes referred herein as a transmission or upload on exception. A determination as to whether to transmit or upload on exception is made by the processing system(s) of local system based upon preprogrammed rules or protocols. Upon transmission of data to remote system 200, a processing system of remote system 200 may make further determinations, and may, for example, notify a caregiver of the exception.

Depending upon the bandwidth of communication channels between local system 100 and remote system 200, the frequency of uploading collected data to remote system 200 may be increased. Moreover, upon occurrence of certain events such as emergency or exception events, certain data may be uploaded in continuous or substantially continuous manner (for example, in real time). Furthermore, in the case of certain sensor systems (for example, sensor systems to monitor physiological parameters) for certain persons, it may be desirable to increase the frequency of uploads to remote system 200 or to transmit real time data in a continuous or substantially continuous manner in real time to remote system 200 even absent an exception event.

In a number of representative embodiments (as illustrated, for example, in FIGS. 1A through 1C), local system 100 of a monitoring system 50 hereof includes a plurality of sensor systems 110a, 110b, 110c, 110d, 110e, 110f, 110g etc. which communicate using a local network 120 such as a wireless local area network (LAN) with a local data communication device or hub 150. Local system 100 may, for example, be used in connection with a residence, a household, a abode or (generally) a space 10 in the vicinity of person or persons 5. In that regard, plurality of sensor systems 110a, 110b, 110c, 110d, 110e, 110f, 110g etc. may, for example, be operatively connected to or associated with furniture, utilities, equipment, devices, systems or appliances, such as one or more beds 12, ranges 14, refrigerators 16, televisions 18, computers 20, lamps/lights 22 toilets 24, a water utility inlet pipe etc. (see, for example, FIG. 1B). Data from sensor systems 110a, 110b, 110c, 110d, 110e, 110f, 110g etc. of local system 100 (which may be processed at least to some extent in local system 100) may be communicated, transmitted, or uploaded to remote system 200 via, for example, local data communication device 150. Remote system 200 may, for example, include a central processing system or a distributed processing system that may, for example, include one or more computers, servers or server systems 210. Computer(s), server(s) or server system(s) 210 may, for example, include one or more processors or processor systems 212 which are in communicative connection with one or more memory or storage systems 214 as known in the computer arts. Memory system(s) 214 may include one or more databases 216 stored therein. Local system 100 may communicate with a communication system or systems 220 of remote system 200 (for example, via local data communication device 150) through one or more wired or wireless communication channels 300 (for example, landline telephones, wireless telephones, a broadband internet connection and/or other communication channel(s)). Software stored in memory system(s) 214 or in one or more other memory system in communicative connection with processor(s) 210 may be used to process or analyze data from local system 100 and, for example, assist a caregiver with a long-term care plans, alerts, use of additional sensor systems etc.

In a number of embodiments, communication system 220 is in communicative connection with a gateway processor 230 of remote system 200. Gateway processor 230 may, for example, receive data from local data communication device 150 of local system 100, process that data (which may, for example, be received in binary file format) into a format readable by software executed by processor 210, and insert the processed data into database 216. In a number of embodiments, gateway processor 230 is adapted to receive data of a number of different types (for example, data regarding states from sensor systems 110a, 110b, 110c, 110d, 110e, 110f, 110g, data regarding medical device usage, etc.), provide initial processing of such data and route such data into a designated system such as into database 216.

Processing system(s) or server system(s) 210 of remote system 200 receive data from local system 100 and, for example, use/processes the data to implement a long-term care plan. Server system(s) 210 can, for example, apply predetermined rules and/or logic defining alert thresholds, alert methods, appointed caregivers, associated reports for trending etc. in implementing a care plan. Remote alerts can, for example, be activated in the case of predetermined events (or a series or groups of events) or at predetermined levels (as determined by monitoring system 50 on the basis of established rules and/or protocols) so that caregivers can respond in a proactive manner to changes in behavior and/or status of person 5. The alerts can, for example, be dispatched or made available to one or more caregiver (or others) via displays or interfaces in any number of ways through communications channel(s) 300 including, but not limited to interactive voice response or IVR, short message service or SMS, internet web pages, email, other internet communications (for example, instant messaging or IM), and/or smart phone/client applications. Compared to currently available monitoring systems, monitoring systems 50 hereof provide more proactive/timely alerts, while significantly reducing cost and complexity of installation. Caregivers can also transmit inquiries to remote system 200 via one or more communication channels 300 as described above to, for example, inquire of the current “status” of person 5. Such an inquiry may, for example, result in a polling of sensor systems 110a, 110b, 110c, 110d, 110e, 110f, 110g etc. by local data communication device 150 for current or most recent data, which is the uploaded to remote system 200. Further, system 50 can transfer information to third parties (for example, physicians etc.) on the instructions of person 5 as part of an overall care plan. For example, a physician (or other authorized third party) portal can be provided as a module of communication system 220 of remote system 200.

As discussed above, sensor systems 110a, 110b, 110c, 110d, 110e, 110f, 110g etc. of local system 100 may, for example, be used in connection with person(s) 5, space 10, a variety of medical devices, appliances, equipment, utilities etc. to monitor the person's wellbeing by, for example, monitoring activity/inactivity of person 5. Table 1 provides a non-exhaustive listing of a number of representative devices and/or systems that may be monitored and representative sensor types for use in monitoring such devices and/or systems. Information or data can also be garnered from systems external to local system 100 or to space 10. For example, temperature data, weather data etc. can be measured or downloaded from various sources available on networked (for example, via the internet) databases.

TABLE 1

| Device or System Monitored | Representative Sensor Types |
|---|---|
| Medical Devices | Various sensors appropriate to device technology |
| Appliance/Device Current (10 mA-15 A | |
| range) | |
| Appliances (TV) | Current Sensing |
| Appliances (Radio) | Current Sensing |
| Appliances (Computer) | Current Sensing |
| Appliances (Fan, Room AC) | Current Sensing |
| Appliances (Heater, El Blanket) | Current Sensing |
| Appliances (Elec. Toothbrush) | Current Sensing |
| Appliances (Hair Dryer) | Current Sensing |
| Appliances (other) | Current Sensing |
| Appliances (Refrigerator door open) | Ambient Light Sensing, temperature, |
| | current - run-time vs. room temperature |
| | (RF inside metal box) |
| Bed Sensor | Pressure Switch |
| | Accelerometer |
| | Passive IR |
| | Pressurized bladder/Hot Water Bottle |
| | Moisture/Humidity/Wetness |
| Occupancy (Area/Room) | Passive IR |
| | Ambient Light |
| | Acoustic |
| | Microwave |
| | Ultrasonic |
| Kitchen - Oven | IR thermometer |
| | Thermocouple |
| | Current or gas supply |
| | Microwave radiation sensing (2.4 GHz) |
| Kitchen - other | Current sensing (microwave, Fridge, |
| | toaster, coffee maker, other electrical) |
| Phone usage/problem | Off-hook monitor - time delay and general usage |
| | profiling |
| Water (Flow) | Pipe Temperature (absolute & vs. ambient) |
| | Water Level (float in tank) |
| | Ultrasonic flowmeter |
| | Positive displacement flowmeter |
| Water Leakage | Conductivity (water/other liquid on floor) |
| Water Temperature | Thermistor/Silicon, IR, thermocouple, |
| | thermostat |
| | Freeze & scald protection |
| Temperature (room/area) | Local to most/all sensors - |
| | inexpensive to implement, |
| | diagnostics, implicit trending, |
| | correlate with local outside |
| | temperature to assess HVAC operational status |
| Temperature (outdoor) | Temperature sensors - |
| | Information from other external |
| | systems such as web temperature info |
| Doorbell | Acoustic, current |
| Intrusion, Glass Breakage | Acoustic, ultrasonic, microwave |
| Shower | Humidity (delta), optical |
| 230 V systems, high-current systems | Amp clamp or similar isolated current |
| | sensing (DW, dryer, furnace, A/C) |
| Garage door open | Tilt |
| | Ambient Light Sensing |
| Universal interface (I/O - other | ex: door open switches, alarm systems, |
| systems) | HVAC controls, doorbell, 3rd party sensors |
| CO Alarm/Natural Gas Alarm | Electrochemical etc. |
| | Sn-oxide |
| Shock (bottom of steps, other likely fall | Accelerometer acoustic |
| locations) | |
| Walker issues | Tilt |
| | Accelerometer |

As illustrated for representative sensor system 110a in FIG. 1C, sensor systems hereof may include at least one sensing or measuring system 112a, at least one processing system or processor 114a (for example, a microprocessor), at least one a memory system 115a and at least one communication system 116a. Sensor system 112a is adapted or operable to measure one or more variables associated with, for example, a state or change in state of a monitored system. Such states are predefined states or conditions which are dependent upon a system being monitored. Data measured and communicated to local data communication device 150 may, for example, include a time of onset of a state (that is, a time of change from a previous or first state to a latter or second state) and data related to the duration of the state (for example, a time of cessation of a state and/or duration of the state). Processor 114a may, for example, perform operations on data received from sensing system 112a, in a manner predetermined by programming therefor which may be stored in memory system 115a. Processor 114a communicates information or data to communication system 116a, which is adapted or operable to transmit the information or data to, for example, local data communication device 150.

Local data communication device 150 includes at least one communication system 152 which communicates (either unidirectionally or bidirectionally) with communication system 116a of sensor system 110a. In a number of embodiments, each of sensor communication system 116a and communication system 152 includes a wireless transceiver for wireless communication (for example, using a ZIGBEE® or other wireless communication protocol). In the illustrated embodiment, local data communication device 150 further includes one or more processors 154 and one or more memory systems 155. Processor 154 may, for example, be programmed or adapted (via programming stored in memory system 155) to process (or to further process) data from sensor systems 110a, 110b, 110c, 110d, 110e, 110f, 110g etc. Processor 154 may further be programmed or adapted to initiate signals to be transmitted to sensor systems 110a, 110b, 110c, 110d, 110e, 110f, 110g etc. such as wake up signals, data polling signals etc. Moreover, processor 154 may further be programmed or adapted to control communications between one or more communication modules of communication system 152 and one or more modules of communication system 220 of remote system 200. Although a separate local data communication device 150 is provided in a number of embodiments hereof, the functionality of local data communication device 150 can be performed, in whole or in part, by one or more of sensor systems 110a, 110b, 110c, 110d, 110e, 110f, 110g etc.

In a number of currently available monitoring system for various uses, one or more monitoring devices stream analog-based data to a remote or central server or software device which then converts the streamed data to meaningful information. Analog data is by its nature memory intensive and network bandwidth intensive, thereby increasing the cost of transmitting the data, slowing the transmission of the data, and limiting/consuming network bandwidth.

In several embodiments of the methods and systems hereof, plurality of sensors 10a, 10b, 10c, 10d, 10e, 10f, 10g, etc. as described above monitor a set of variables or parameters indicating state(s), changes in state and/or a lack of a change in state (for example, indicating operational use or disuse) of, for example, household devices or systems, household appliances, utilities (for example, water, electricity, sewage, gas, fuel oil etc.), furniture (or example, beds, chairs etc.) medical devices and/or any other devices or systems. Sensors 10a, 10b, 10c, 10d, 10e, 10f, 10g, etc. collect analog data which are recorded (or convert into) event or state-based data, which can be represented as discrete values. Data of states and changes of states (as defined in monitoring system 50) of a monitored device or system may, for example, be generated to provide a state history in which, for example, defined states and durations of such defined states over time are set forth for a period of time. Rather than transmitting a stream of analog operational or status data, state-based data or values which, for example, correspond to the state or state history of a monitored device or system (for example, time of use/state change, duration of state, level of use etc.) for a period of time are transmitted in a noncontinuous, discontinuous or batch manner at intervals spaced in time (although not necessarily at regularly spaced intervals) to communication system 20 of remote system 200. In that regard, the data may be transmitted by communication system 152 of local data communication device 150 via one or more of communication channels 300 (for example, via telephone, internet etc.) to communication system 220 of remote system 200. The data may, for example, be transferred periodically (for example, hourly, daily etc.). Different data or values may, for example, be transmitted with different time intervals or frequencies depending upon the nature of the underlying event(s) or values as set forth in predetermined rules.

As described above, some processing of data occurs in a processing system of local system 100. Such processing may, for example, occur in a processor or processors of one or more of sensors 10a, 10b, 10c, 10d, 10e, 10f, 10g, etc. (for example, in processor 114a of sensor system 110a), in a processor or processors 154 of local data communication device 150 and/or in one or more other processors of local system 100 before transfer of data to the remote system 200. In a number of embodiments, local data communication device 150 serves as a repository for all information coming from sensors 10a, 10b, 10c, 10d, 10e, 10f, 10g, etc. Additional processing in processor 154, when effected, may, for example, include: comparing of values with prior average values, evaluation of combinatorial events from more than one sensor or sensor system to infer or determine situations or events not necessarily inferable or determinable from a single sensor or sensor system, and the transmission of data/information to remote system 200. In that regard, a plurality of sensors working in concert as part of a larger network monitoring system and designed to upload data on, for example, a predetermined period leave open the possibility that a meaningful event can occur in space 10 that does not generate an alert or alerts from remote system 200 until the data is uploaded to remote system 200. This delay can reduce the effectiveness of monitoring system 50 and potentially result in negative clinical benefits to person 5 if it results in delay of an appropriate reaction to a clinical need or problem. Continuous streaming of analog data may prevent such negative clinical outcomes, however, as described above, transmission of real time streams of monitored data is expensive, requires substantial network bandwidth and requires a substantial amount of memory.

In a number of embodiments, transmission of data to remote system 200 occurs on a regular, periodic basis and/or on an unscheduled or exception basis. In that regard, exceptions or triggering events defined by predetermined states or state changes, groups of states or state changes, events, thresholds, or business logic, are established which, when determined to be in existence (using defined rules), trigger an automatic upload of data to remote system 200 regardless of predetermined upload cycles. Such exceptions or triggering events result in more timely and effective monitoring of person 5. Software or logic to determine such an exception or a triggering event can, for example, be resident on a sensor system, on local data communication device 150 and/or on a separate processor system of local system 10. Thus, an exception occurs when a condition is determined to exists (via processing/analysis of sensor data in local system 100) which requires expedited or immediate attention from remote system 200.

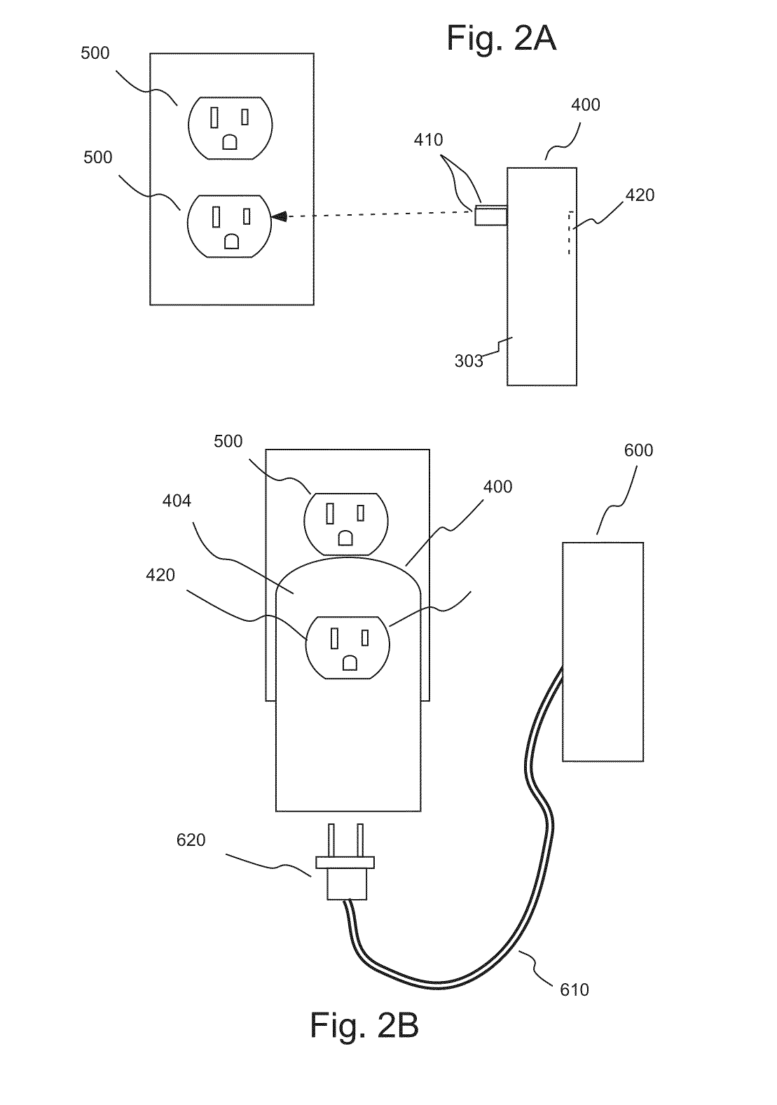

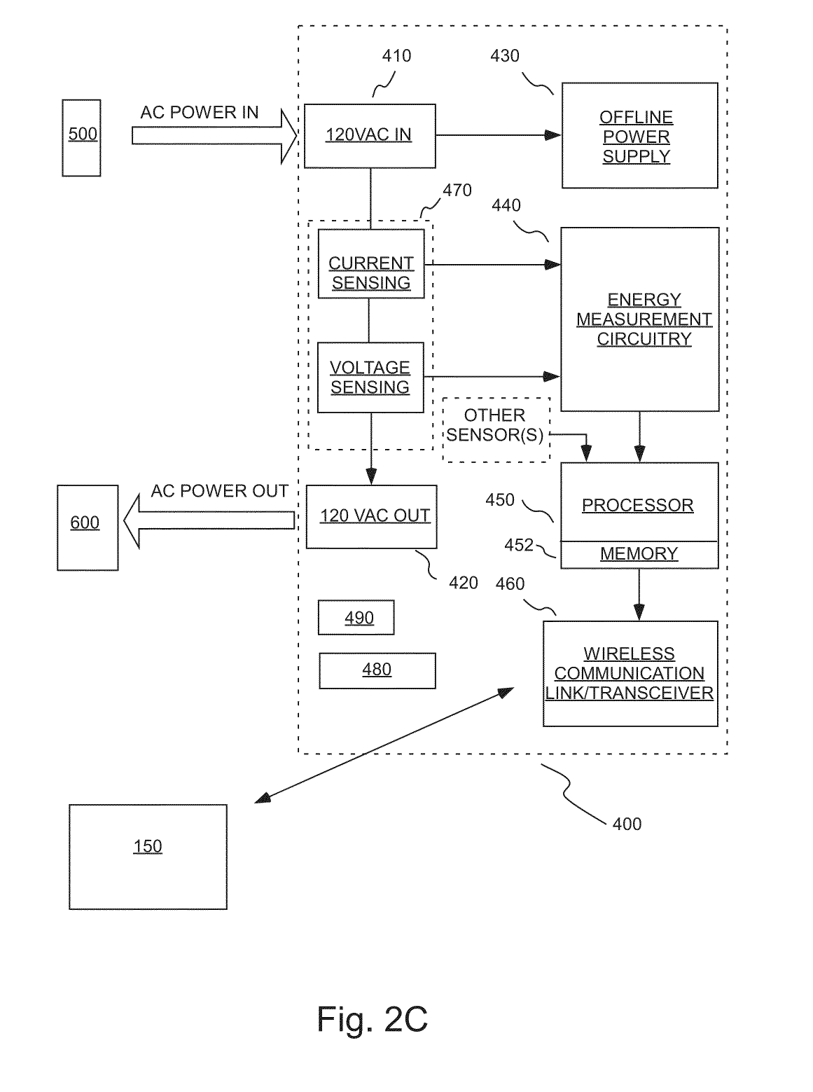

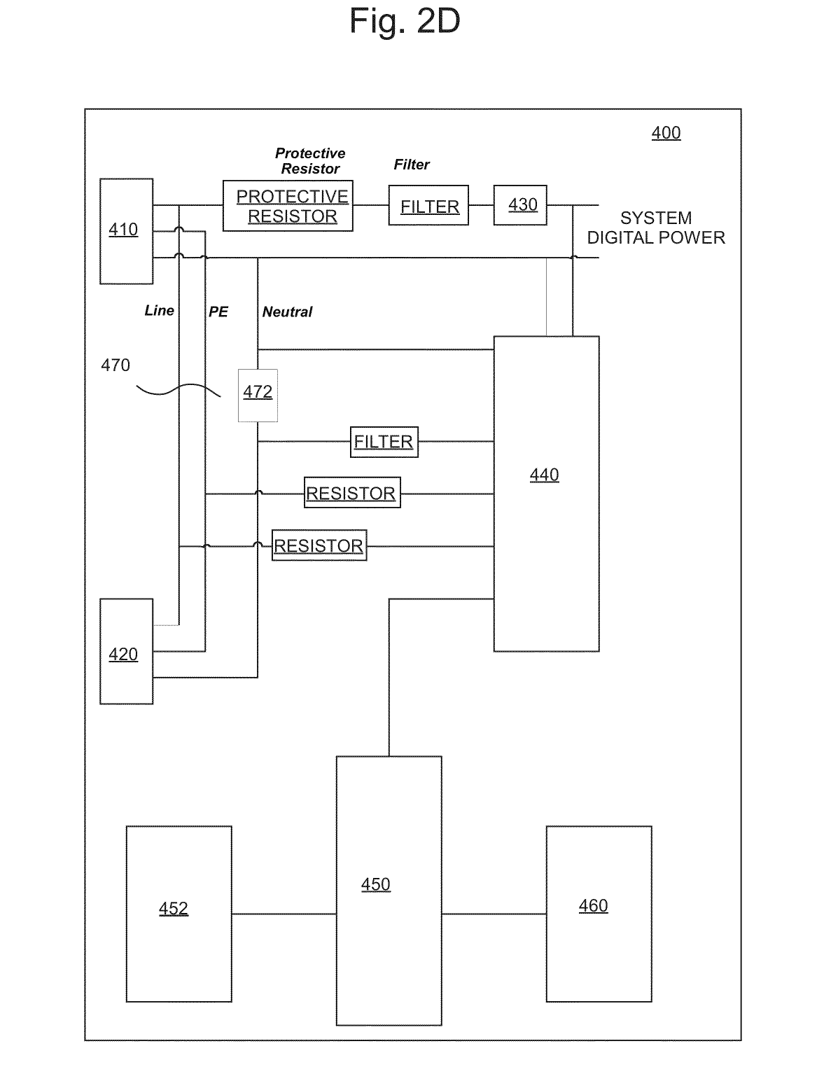

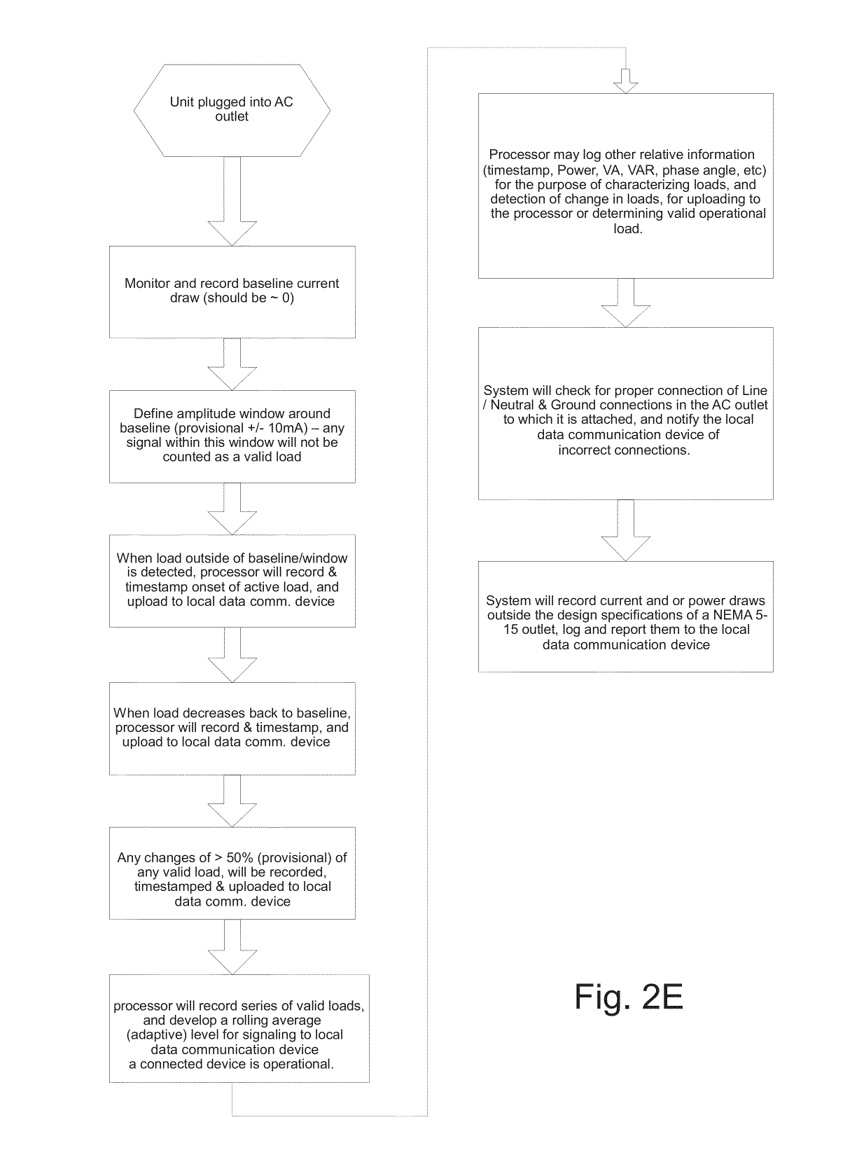

Several types of representative sensor systems for use in the systems hereof are discussed in further detail below. One type of sensor system used in the systems hereof is an energy sensor system that can be used in connection with electrically powered devices attached to an electrical outlet in space 10. One or a plurality of sensor systems 10a, 10b, 10c, 10d, 10e, 10f, 10g, etc. may be an energy sensor system as describe herein. A representative embodiment of a modular or universal energy sensor system 400 for use with electrically powered devices is, for example, illustrated in FIGS. 2A through 2D. Energy sensor system 400 can, for example, be used in connection with monitoring any one of many electrically powered devices (for example, televisions, radios, computers, kitchen appliance, other appliances etc.). For example, energy sensor system 400 can be used in connection with an device or system operating within a defined range of voltages and/or a defined range or currents. Energy sensor system 400 may, for example, be plugged into a standard NEMA wall power outlet or receptacle 500 via plug contacts 410 extending from a rearward surface of a housing 404 of energy sensor system 400. Energy sensor system 400 may also include a standard NEMA outlet 420 to receive a standard NEMA plug 620 of a power cord 610 from a monitored device 600 (see FIG. 2B), such that the current flows through the circuitry (see FIGS. 2C and 2D) of energy sensor system 400. The existence, magnitude, phase angle, voltage etc. of current draw through power cord 610 indicates, for example, that monitored device 600 is in use, the duration of use, the nature of the use etc.

Energy sensor system 400 may, for example, be standardized for universal use in connection with devices using, for example, 110 volt power. As described above, energy sensor system 400 may be plugged into any standard household AC outlet, socket or receptacle 500, and may receive standard NEMA 5-15 power plug 620 from cord 610 connected to any device 600 to be monitored. As illustrated in FIG. 2C, AC power is supplied to energy sensor system 400 via, for example, standard NEMA 5-15 power plug 410. AC power is supplied from energy sensor 400 to monitored device 600 via, for example, standard NEMA 5-15 power socket 420.

Power for the circuitry of energy sensor system 400 may, for example, be derived from an off-line switching power supply 430. Power supply 430 may, for example, include an integrated circuit, IC or chip such as a Linkswitch LNK-305 series IC available from Power Integrations of San Jose, Calif. and associated passive components, which generate a voltage of, for example, −3.3 VDC with respect to an AC neutral line. In the illustrated embodiment, power supply 430 powers an energy monitoring chip 440, a computer processor 450 (for example, a microprocessor) and a wireless communication link or module 460.

A sensor 470 may, for example, include a low value (for example, 0.004Ω nominal) series ohmic shunt 472 which is placed in series with the neutral connection between NEMA input plug 410 and NEMA output socket/outlet 420, to which monitored device 600 is connected. A voltage is developed across the shunt resistor, which is proportional to the current flowing through it. The measured current may be used for the calculation of current draw, power, and/or other parameters of monitored device 600. Voltage sensing of the AC circuit being monitored may, for example, be accomplished via a network of high-value resistors which are connected to line, neutral and ground. The measured voltage may be used to determine voltage, phase angle, power factor and other parameters of interest of the power source and effects thereon by the connected load.

In a number of embodiments, a Maxim 78M6612 power and energy measurement integrated circuit, chip or system-on-a-chip available from Maxim Integrated Products, Inc. of Sunnyvale, Calif., monitored the voltage and current delivered to monitored device 600 through the above-described electrical networks, and processed the information to generate digital information including, but not limited to, AC voltage, current, power, VA, phase angle and other parameters which characterize the operational status or state of monitored device 600. Operation of the Maxim 78M6612 power and energy measurement integrated circuit is described in the 78M6612 Single-Phase, Dual-Outlet Power and Energy Measurement IC Data Sheet, Maxim Integrated Products, Inc. (June 2009), the disclosure of which is incorporated herein by reference. Processor 450 is, for example, a Microchip PIC-series PIC24FJ128GA006-I/PT microprocessor available from Microchip Technology, Inc. of Chandler, Ariz. Operation of the Microchip PIC-series PIC24FJ128GA006-I/PT microprocessor is described in the PIC24FJ128GA010 Family Data Sheet, Microchip Technology, Inc. (2009), the disclosure of which is incorporated herein by reference. Processor 450, may for example, perform operations on the electrical data received from the energy monitoring chip 440, as, for example, specified in the operational description below and the flowchart of FIG. 2E. Processor 450 relays information via, for example, wireless communication link 460 (which may, for example, be an RF connection using, for example, Zigbee protocol) to local data communication device 150. A wireless RF communication connection may, for example, be established via a Microchip MRF24J40MA-I/RM Zigbee module available from Microchip Technology, Inc., which is controlled by processor 450. Operation of the Microchip MRF24J40MA-I/RM system is described in MRF24J40MA Data Sheet, Microchip Technology, Inc. (2008), the disclosure of which is incorporated herein by reference. Communication link 460, for example, uploads the information derived from energy monitoring chip 440 under control of processor 450, in accordance with defined variable changes corresponding to defined changes of state of monitored device 600. Sensor or sensing circuitry 470, wireless communication link 460, processor 450, energy measurement circuitry 440, and power supply 430 are integrated into a single unit within housing 404.

Devices such as monitored device 600 may, for example, operate from a nominal VAC source, and may, for example, be limited in current draw to approximately 15 A. In a number of embodiments, the minimum current draw which may be resolved is approximately 0.010 A. One or more indicators 480 (see FIG. 2C) such as one or more lights may be provided to indicate different operational states of energy sensor 400, including, but not limited to, communication (RF) pairing, ready for operation, power available and/or fault status. A switch 490 (see FIG. 2C) may be provided for a user to, for example, initiate an RF pairing process, wherein energy sensor 400 is associated with a specific central data collection point or local data communication device 150 operating on the same RF channel. Switch 490 may, for example, be mechanical, magnetic or operated by other inputs. Energy sensor system 400 may, for example, include one or more magnetic reed, capacitive or other switches for the purpose of performing various functions, including, but not limited to the initiation of RF pairing operations.

As set forth in the flowchart of FIG. 2E, energy sensor system 400 may, for example, monitor and record a baseline current draw (for example, approximately 0 A in a number of devices). As described above, an amplitude window around the baseline (such as approximately +/−0.010 A or 10 mA) may be defined. Any signal within the defined amplitude window will not be considered a valid load. When a load is outside of the baseline window is detected, processor 450 may, for example, record and timestamps the onset of the measured current/active load. This information may be uploaded to local data communication device 150. When the measured load decreases to the baseline load, processor 450 records and timestamps the decrease in load. This information may also be uploaded to local data communication device 150. In a number of embodiments, any changes of a certain threshold (for example, 50% or greater) of any valid load are recorded, time-stamped and uploaded to local data communication device 150. Processor 450 may, for example, record a series of valid loads and develop a rolling average (adaptive) level for signaling to local data communication device 150 that monitored device 600 or other connected device is operational. As described above, processor 450 may log other relevant information (for example, timestamp, power, VA, VAR, phase angle, etc.) to characterize loads and detection of changes in loads for uploading to local data communication device 150 and/or for determining valid operational load.

Energy sensor system 400 is adapted to or operable to monitor an unknown variety of devices which may, for example, be found in space 10 (for example, a home). Because of this uncertainty regarding the status of a device in terms of, for example, current draw during various states (for example, when “on”, “asleep”, “off” or in another mode or state), energy sensor system 400 monitors various current or power draws of the device over a predetermined period (for example, in the range of approximately 3-7 days). As energy sensor system 400 monitors the power or current draw of the connected/monitored device, it may, for example, record minima and maxima of those values. From the minima and maxima data points, a reference in between those points may be generated or determined that is set as the decision point for determining whether a device is, for example, in an “on” state, in an “off” state or in another defined state. This methodology is in contrast a methodology in which a fixed threshold is established for determining operational status or state. Many devices continue to draw current even while in an “off” state (in terms of the user's perception) and any preset or fixed threshold runs the risk of incorrectly determining the status of a connected device. Energy sensor system 400 continuously record and updates the determined threshold, making energy sensor system 400 usable even if the connected device is changed.

In a number of embodiments, after a device such as device 600 is connected to energy sensor system 400 and a nonzero load is detected, energy sensor system 400 begins recording measured current values. After a defined period (for example, 72 hours), energy sensor system 600 may, for example, determine the standard deviation of the measured values, and, if exceeding a preset or determined amount, average the group of values in the high range, and average the group of values in the low range. Energy sensor system 600 may then establish a threshold using an equation such as, for example, avg low+(avg high−avg low)/5 or a similar equation, and use the calculated threshold to determine and record states (for example, on or off states). As the values are continuously recorded, the averages and determined threshold may update, so that energy sensor system 400 dynamically adapts.

In general, energy sensor system 400 may send status of electrical power, and/or status of monitored device 600 in real-time to local data communication device 150, or timestamp and store such or similar information for transmission at a predetermined or externally requested time. With respect to the status of electrical power, energy sensor system 400 can readily detect an incipient loss of power and transmit data regarding such an event to local data communication device 150. Likewise, energy sensor system 400 can detect resumption of interrupted power and transmit such data.

Energy sensor system 400 may also check for proper connection of the line, neutral and ground connections in AC outlet 500 to which it is attached and notify local data communication device 150 or incorrect connections. Energy sensor system 400 may also record current, power draws and/or other measure variables outside the design specifications of a NEMA 5-15 (or other specified) outlet and log and report such information or data to local data communication system 150.

In the systems and methods hereof, use of a monitoring technology to track usage of a variety of household electrical items and/or appliances is simplified with the use of a universal sensor system such as energy sensor system 400. Because energy sensor system 400 may be used in connection with more than one type of device, the identification of the device being monitored may be desirable.

If the device being monitored is assigned or identified incorrectly, false positives or negatives in uploads on exception and/or alerts generated by remote system 200 may result, thereby reducing the effectiveness of monitoring system 50 in monitoring the wellbeing of person 5 Energy sensor system 400 and/or other universal sensor system may, for example, be provided with a selector via which person 5, a caregiver, an installer or other person identifies the type of device to which the sensor system is attached. However, such a selector leaves open the possibility of human error.

Processor 450 of energy sensor system 400 (and/or one or more processors in communication with energy sensor system 400) may, for example, use the existence of unique current draw and/or other characteristics to determine if energy sensor system 400 is being used in connection with a particular device or system. Processing system 450 of energy sensor system 400 may, for example, execute one or more algorithms to determines operational status of a connected device. Each monitored device or system has unique current draw and/or other electrical characteristics which may be used to either identify the device or system, or, at a minimum, rule out certain other possibilities. Examples of parameters to be monitored to determine an attached device include current frequency, current amplitude, phase angle, Fourier transform pattern, real power, reactive power, imaginary power, power factor etc. Algorithms to identify and/or monitor a device may, for example, consider sleeping modes or states, energy saving modes or states, etc. and dynamically adapt to different devices automatically. Operating and/or non-operating electrical characteristics of a monitored device or system can, for example, be compared characteristics of known electrical devices or systems for the purpose of determining or inferring the type or nature of an otherwise unknown connected device. Stored equations or look-up tables of known electrical device characteristics can, for example, be stored in memory system 452 of energy sensor system 400 or in a memory system in communicative connection with energy sensor system 400 for comparison to measured characteristics of a monitored device or system.

After determination of the type, nature or identity of a connected/monitored device, a logic check can, for example, be performed to ensure that current draw and/or other characteristics are consistent with the device assigned to a given monitor. If the current draw and/or other characteristics do not match the assigned device, the associated data can, for example, be flagged as suspect. Such a device recognition system can, for example, reduce errors and simplify installation. The logic check can, for example, using a processing system of local system 100 and/or a processing system of remote system 200 (for example, using energy sensor system 400, local data communication device 150, server system 210 and/or another processing system).

Variables other than can energy-related variables can, for example, be monitored by energy sensor system 400 via one or more other sensors (illustrated schematically in FIG. 2C). For example, energy sensor system can also include one or more other sensors to monitor environmental signals such as ambient light, motion, acoustic noise, temperature, humidity and/or other environmental conditions. Such conditions can, for example, effect current- or other energy-related variables measured by energy sensor system 400 and may, for example, be used for the evaluation of circuit performance or ambient environmental conditions and/or correction of measured energy-related variables.

In the case of devices or appliances that use current other than 110 volt current (for example, an electric range), a sensor system other than energy sensor system 400 may be used. For example, an impedance sensor system may be used to measure or determine states, changes of state etc. For example, a current sensitive/impedance sensor system can be placed in operative connection with (for example, fit around) the power cord of the electric range or other device. The existence of current draw through the power cord will, for example, indicate that the range or other device/system is in use, and for what duration.

In the case of a number of devices, changes in state secondary to the primary function of the device (for example, from one or more subsystems of the device) can be monitored to measure changes in state of the device. To monitor refrigerator usage, for example, a light-sensitive sensor system or a current- or energy-based sensor system (for example, as described above) in electrical connection with the refrigerator/refrigerator light bulb may be used to monitor state changes of the refrigerator. For example, a current sensitive sensor system may be used in connection with the electrical outlet of the refrigerator light. The existence of current draw through the refrigerator light bulb indicates that the refrigerator door has been opened, and for what duration.

Various sensor systems can also be used to measure utility usage such as water, heating and air conditioning, sewage etc. By, for example, measuring the water intake of a household (or other abode) at the input pipe of the household, a remote caregiver has the ability to track water usage associated with monitored person 5 using the bathroom, taking showers, washing dishes, washing clothes, etc. These behaviors are, in part, an indication of the wellbeing of monitored person 5.

Water consumption can, for example, be measured using a variety of methods including, for example, a mass flow sensor system that clips around the intake pipe of the household water supply and senses water flow and/or water volume consumed, a temperature sensor system that senses temperatures different than room temperature as well as other methods. Water consumption can also, for example, be measure using an acoustic sensor as described in U.S. Provisional Patent Application Ser. No. 61/541,444, filed Sep. 30, 2011, the disclosure of which is incorporated herein by reference.

One or more sensor systems can, for example, be used to measure one or more variables related to rest and/or sleep (for example, the duration of time that monitored person 5 is lying in bed, sitting in a chair, sitting on a sofa etc.), which are important parameters for monitoring the wellbeing of person 5. In addition to the duration of time spent in bed, the time of going to bed, the time of waking up and the time and duration of interruptions of sleep (such as associated with the use of the restroom in the middle of the night), may also be recorded. Failure to get out of bed by a certain time, for example, may be indicative of a problem requiring immediate attention (and defined as an exception event required an expedited or immediate upload of data to remote system 200).

Monitoring of bed usage can, for example, be accomplished in various manners including, for example, use of a pressure sensitive member (for example, a pad, tube etc.) placed on or under the mattress of the bed to indicate the presence of a person in bed, or the use of a pressure sensor located on or under a leg of the bed and designed to monitor change in weight, thereby indicating the presence of a person in bed. Other sensor systems for sensing the presence of a person in a bed may, for example, include piezo resistive films, thick film strain sensors, infrared sensors, accelerometers, acoustic sensors, carbon dioxide sensors and/or body temperature sensors.

In a number of embodiments, one or more sensor systems provides for detection of the presence and/or movement of person 5 on an item or an object such as an item of furniture (for example, items of furniture upon which person 5 would rest including, but not limited to, a bed, a chair, a sofa etc.). Such sensors are referred to herein generally as presence sensor systems. Presence sensor systems hereof may, for example, include one or more enclosed sensor volumes filled with a flowable fluid (for example, a flowable liquid and/or a gas). The enclosed sensor volume(s) may, for example, be enclosed by an extending member. In a number of embodiments, the extending member includes an outer layer or surface which encloses the fluid such that the sensor volume changes or compresses, resulting in a change in pressure, upon an applied external force or pressure but recovers or substantially recovers to an initial or baseline volume when the external force or pressure is removed. The outer layer or surface may, for example, have resilient properties or one or more resilient members within the sensor volume may provide resilience.

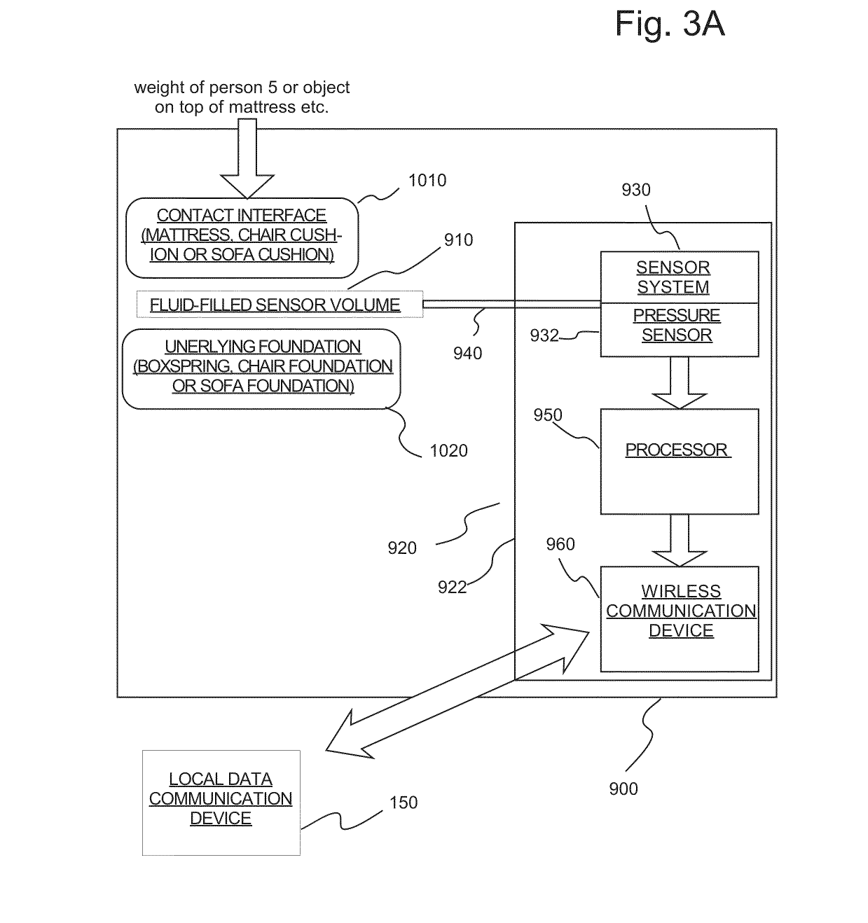

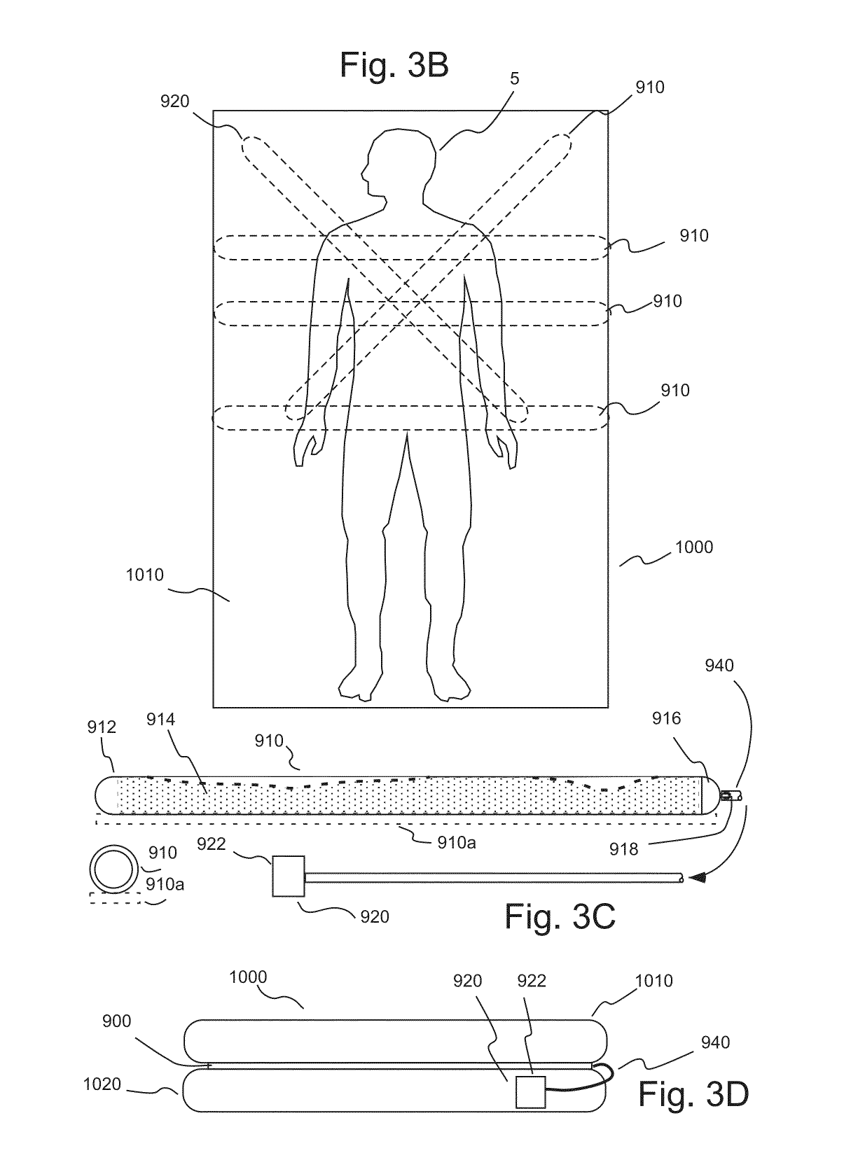

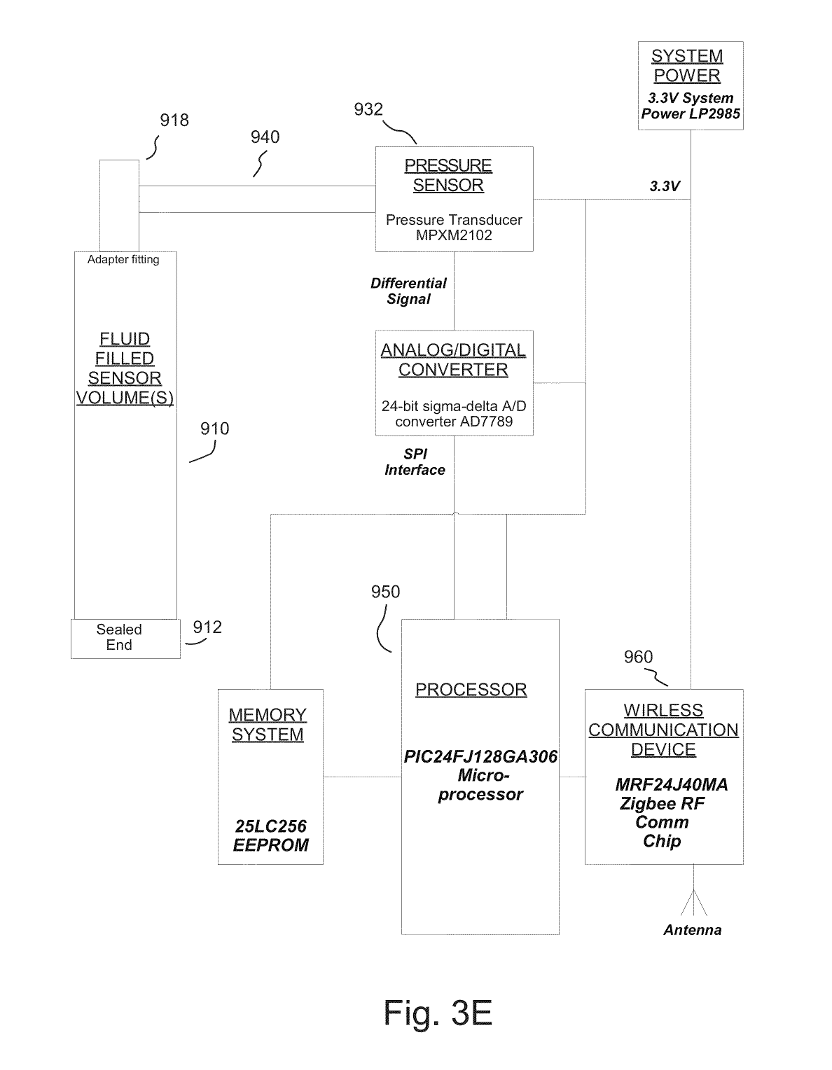

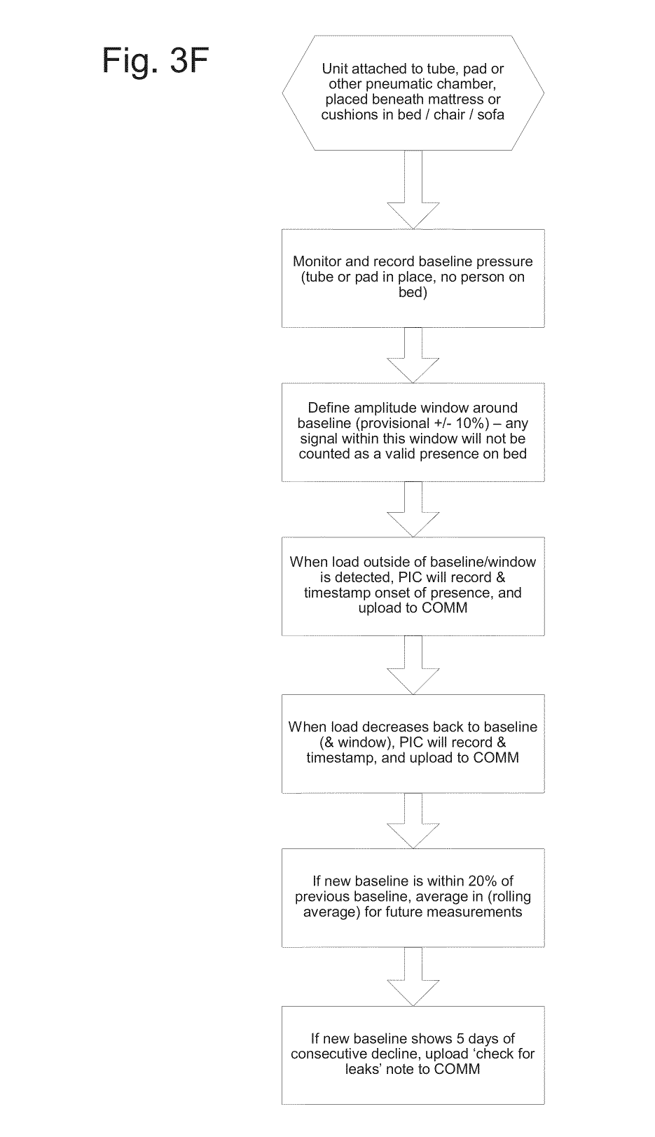

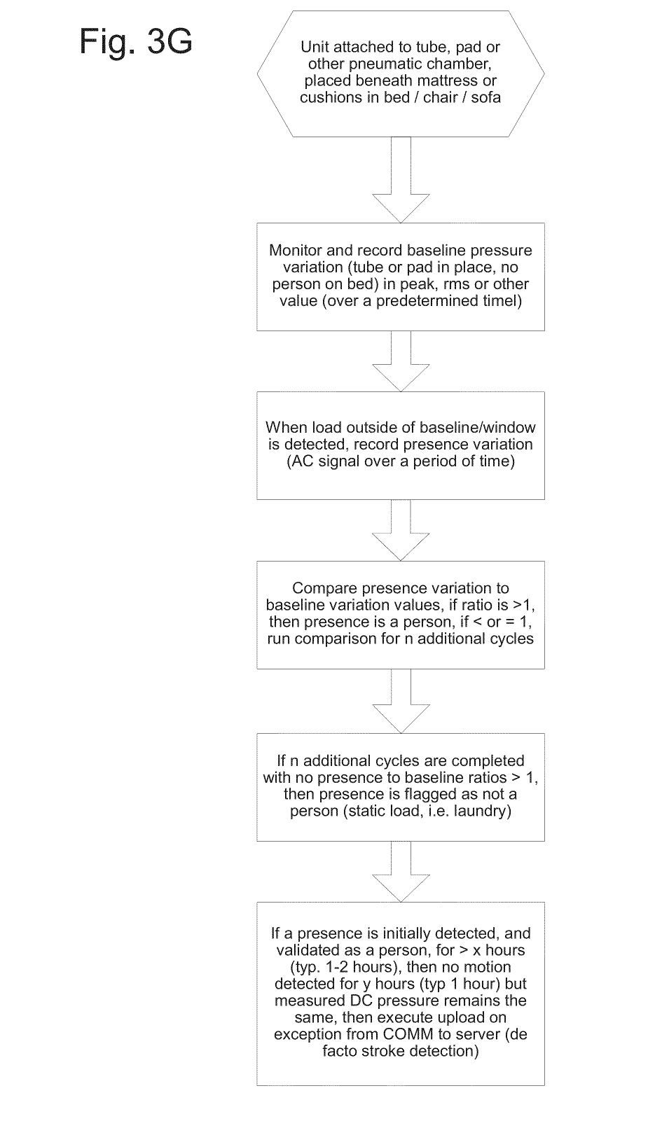

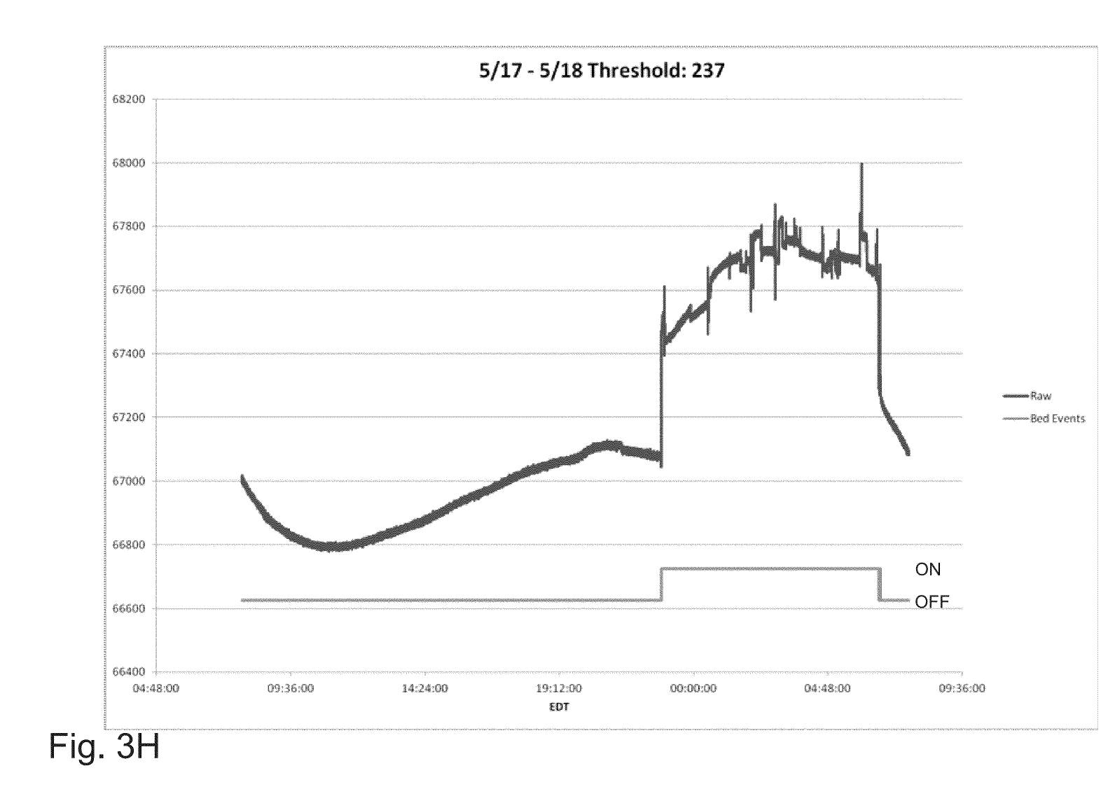

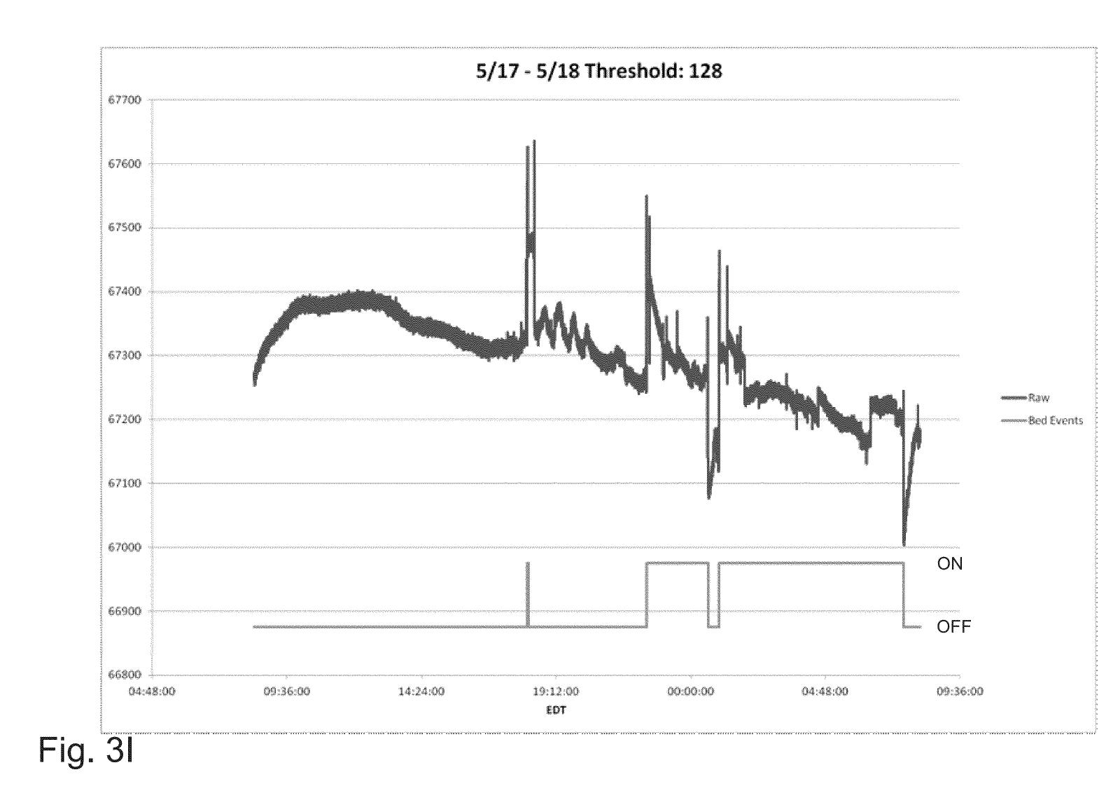

In the embodiment illustrated in FIGS. 3B through 3D, presence sensor system 900 includes one or more fluid-filled structures having a certain resiliency or spring rate. In a number of embodiments, presence sensor system 900 includes a single extending fluid-filled member or structure 910 which may, for example, be oriented in one of the orientations relative to a bed 1000 illustrated in FIG. 3B. As illustrated in FIG. 3D, presence sensor system may, for example, be placed between a mattress 1010 and a box spring 1020. In a number of embodiments, presence sensor system 900 may include a plurality of extending members 910. Extending member or members 910 form a compressible, sealed sensing volume underneath a position on an object where person may be present (for example, directly upon or underneath pads, mattresses or other cushioning or coverings of beds, chairs, sofas etc.). Extending member or members 910 can form a sensor volume of any shape, including, for example, tubes (either straight or curved) pads, etc.

Algorithms were developed to detect a person in bed and generate corresponding “on” (presence) and “off” (absence) events. In a number of embodiments, presence sensor system 900 uses an absolute pressure sensor measuring pressure changes in sealed extending member(s) 910. Algorithms for present detection may, for example, adapt to the “system” configuration and/or conditions and reject, for example, ambient changes in the environment.

In a number of embodiments, compression of or more extending member 910 conveys pressure changes and other signals (for example, a flow signal) resulting from forces associated with presence and/or movement of person 5 or an object positioned on top of the extending member 910, or an interposing pad, to a unit 920 remote from extending member 910 via an intermediate connector 930 (for example, flexible conduit). Unit 920 may, for example, include a sensor system 930 having one or more sensors including, for example, a pressure sensor or pressure transducer 932, a flow sensor, etc. within a housing 922.

Variables impacting presence sensor system 900 may, for example, be broken down into categories including: environmental, sensor system configuration, and system load. Presence sensor system 900 reacts, for example, to environmental temperature and barometric pressure changes. Such changes are detected by presence sensor system 900 as relatively slow changes that occur over periods of tens of minutes to hours and days. Temperature changes may, for example, be induced by a heating, ventilation and air conditioning or HVAC system. Presence sensor system 900 is also impacted by the extending member or members 910. For example, rigidity, which is determined by, for example, material selection and wall thickness, impacts the level of signal. Rigidity may vary from device to device. The load includes, for example, mattress 1010 in the case of bed 100. Construction and configuration of mattress 1010 impacts the weight and load placed on the extending member 910. A person's sleeping position also effect the load detected by presence sensing system 900. These positions include, for example, sleeping on the back, front or side, all of which effect the weight/weight distribution placed over extending member 910.

The sensor volume within extending member 910 may, for example, form an air-filled chamber wherein the air within the chamber or sensing volume of extending member 910 is at (or approximately at) ambient or atmospheric pressure. Maintaining the fluid within the sensing volume at or near ambient pressure reduces the likelihood of leakage as compared to a system in which the sensing volume is pressurized to be at a pressure above ambient pressure. As describe above, outer layer or surface 912 of extending member 910 may, for example, be resilient and/or one or more resilient members 914 (for example, formed of a resilient material or materials and having substantial void volume) may be positioned within the sensor volume to provide resilience. In a number of embodiments, extending member 910 was formed from a ¾″ diameter length of flexible polyvinylchloride or PVC tubing or silicon tubing having a length of approximately 4 feet (for use, for example, in connection with a bed) that was sealed at one end. The other end of extending member 910 included a seal member or plug 916, which sealed to the tube wall via, for example, an interference fit. Sealing member 916 may, for example, include a fitting 918 (for example, a barbed fitting) which is open to or in fluid connection with the sensor volume with extending member 910, and permits intermediate connector 940 (for example, a length of flexible tubing) to be affixed to and in fluid connection with the sensing volume. In that regard, intermediate connector is also in fluid connection with external or remote unit 940 so that pressure sensor 932 or transducer and/or one or more other sensor can measure the pressure within the sensing volume and monitor pressure and/or flow resulting the presence of a person 5 and/or movement associated with the presence of person 5.

In a number of embodiments, one or more extending member or members 910 includes an extending “lower” or “bottom” support section or member 910a positioned on the side or area of extending member 910 generally opposite the side or area closest to placement of the load to be detected (shown in broken or dashed lines in FIG. 3C). Section 910a is more rigid than resilient layer 912 and provides support in uses wherein, for example, an adequate support is not provided below the placement of extending member 910. Such rigidity may, for example, be provided by the material characteristic of extending section 910a and/or the dimension(s) thereof. Such an embodiment may, for example, be useful in connection with certain “hospital” beds which do not include a box spring or similar component. In a number of embodiments, section 910a was formed generally integrally or monolithically with outer layer 912 in a polymeric co-extrusion process. Such components may alternatively be formed separately and attached. As clear to one skilled in the art, many different fabrication methodologies may be used.

In the case that presence sensor system 900 includes a plurality of extending members 910, sealing members 916 thereof may, for example, be in fluid connection with a manifold system (not shown), which is in fluid connection with remote unit 940 so that a single pressure sensor can monitor pressure. Alternatively, each of a plurality of extending members 910 can be in fluid connection with a separate pressure sensor which may, for example, be housed within remote unit 940.

In a number of embodiments, intermediate connector 940 was formed from flexible polyvinylchloride tubing having a the length approximately 6 feet and a diameter less than the diameter of extending member 910. In a number of such embodiments, intermediate connector had a diameter of approximately ¼ inch. The length of intermediate connector 940 may, for example, be selected to obtain specific temporal response characteristics—for example, by changing overall system volume and time constants, as dictated by specific requirements.

Pressure sensor 932 may, for example, include an absolute pressure transducer with a provisional range of 10-110 kPa. In a number of embodiments, the pressure transducer was a Freescale MPXM2102AS available from Freescale Semiconductors, Inc. of Austin, Tex., which is an on-chip, temperature compensated silicon pressure sensor with a nominal range of 10-110 kPa.

A block circuit diagram of an embodiment of the electronics of presence sensor system 900 is illustrated in FIG. 3E. In the illustrated embodiment, the pressure transducer differential output is connected directly to a 24-bit A/D converter (for example, an AD7789 A/D converter, available from Analog Devices, Inc. of Norwood, Mass.) in a ratiometric configuration, with the bridge supply and reference voltage for the A/D converter being electrically the same, which eliminates drift issues with separate references. The Analog to Digital (A/D) converter is connected to processor 950 such as a microprocessor (in a number of embodiments a PIC series microprocessor such as a Microchip PIC24FJ128GA006-I/PT microprocessor available from Microchip Technology Inc. of Chandler, Ariz.) via a (SPI/I2C) interface.

Processor 950 performs operations on the pressure data received from the A/D converter, as, for example, set forth in the operational description and flowcharts of, for example, FIGS. 3F and 3G. Processor 950 transmits data/information via a wireless communication device 960 (for example, including an RF transceiver and ZIGBEE protocol) to local data communication device 150. In a number of embodiments, the RF transceiver was a MRF24J40MA-I/RM Zigbee module available from Microchip Technology, Inc., which was controlled by processor 950.

FIG. 3F illustrates an embodiment of a flowchart for a methodology of determining the presence of person 5 or another person on an item of furniture such as bed 1000. As described above, one or more fluid-filled sensing volumes such as extending members 910 is first positioned, for example, beneath mattress 1010 of bed 1000. In a number of embodiments, a measuring or sensing algorithm measures the change between two pressure values and compares the change against a threshold. The compared pressure values may, for example, be averages and the threshold may be adjusted for presence sensor system 900 and it environment of operation. Sensor system 930 monitors and records baseline measurements (for example, a baseline pressure via pressure sensor 932) wherein no person in present on the bed. An amplitude around the baseline (for example, +/−10%) is defined. Any signal within the defined amplitude window will not be determined as a valid presence on bed 1000. When a pressure is measured outside of the baseline window, processor 950 will record and timestamp the measurement as the onset of presence. When the measured pressure decreases back to within the baseline window, processor 950 will record and timestamp the measurement as an end of presence. Onset of presence and end of presence may, for example, be transmitted to local data communication device 150 upon determination or may be stored by sensor system 900 and transmitted at a later time.

If a newly determined baseline is within a certain percentage (for example, within 20%) of the previously determined baseline, the new baseline may, for example, be averaged with previous baseline measurement to create, for example, a rolling average for future measurements. If a new baseline is not within 20% of the previous baseline, it may indicate a problem (for example, a leak in the sealed sensing volume). Likewise, if newly measured baselines exhibit a decreasing trend for a certain period (for example, for 5 days), this may indicate a leak. In such conditions, processor 950 may, for example, cause communicate a signal to local data communication device 150 to cause an upload to remote system 200 to cause an alert or a notice to check for leaks in the sensing volume of presence sensor system 900 or one or more other problems.

FIG. 3G illustrates an embodiment of a methodology of operating presence sensor system 900 for detection of motion of person 5 or another person after sensing presence as described above. Sensing of motion may, for example, be used to validate that a person rather than an inanimate object is resting on an item or to monitor status of a person well present on the item (for example, a bed). As set forth in FIG. 3G, sensor system 900 may, for example, monitor and record baseline pressure variation by measuring peak pressure, root mean square (rms) pressure and/or other value(s) over a predetermined period of time. When a load outside the baseline window is measure as described above, an onset of presence is determined Variation in the load (for example, as measure by pressure variation) is measured over time. The measured variation during pressure may be compared to baseline variation values. In a number of embodiments, if a ratio of >1 is determined, this determination is associated with validation of the presence of a person. If the ratio is <1, the comparison may, for example, be run for n additional cycles, wherein n is an integer. If n additional cycles are completed with no presence to baseline ratios >1, the presence is flagged as not a person (that is, as a static load such as laundry etc.).

In a number of embodiments, if presence is initially detected and validated as a person for a certain period of time (for example, greater than x wherein x is, for example, 1-2 hours), and, subsequently no motion is detected for a define period (for example, greater than y wherein y is, for example, 1 hour), but measured DC pressure remains the same, this measured lack of motion may be indicative of an emergency condition of person 5 and an upload on exception from local data communication device 150 to remote system 200 may be initiated.

In a number of embodiments, average upper and lower (or average minimum and maximum) pressure measurement are determined by presence sensor system 900 and adaptively updated as, for example, described above. For example, over a period of time (for example, several days) the system 900 monitors and/or records pressure and determines a minimum/lowest average pressure and a maximum/highest average pressure. In a number of embodiments, a threshold was mathematically derived to distinguish between presence of person 5 (or another person) and absence of person 5 (or another person). In a representative example, a threshold change was determined (for example, via processor 950) as a percent of the delta or difference between the minimum average pressure and the maximum average pressure (for example, x %*(max-min)). In such a representative embodiment, if the minimum average pressure was, for example, determined to be 1 psi and a maximum average pressure was, for example, determined to be 10 psi, the threshold change would be 0.2*(10-1) or 1.8 psi. The threshold or decision point between presence and absence would be determined as the minimum average pressure plus the threshold change or 1 psi+1.8 psi=2.8 psi. Thus, 2.8 psi would be set as the threshold or decision point, wherein a pressure less than 2.8 psi would be determined to correspond to absence, and a pressure of greater than 2.8 psi would be determined to correspond to presence. Some variation or hysteresis may, for example, be provided around the threshold pressure before a change of state is determined (for example, 0.5 psi). As clear one skilled in the art from the disclosure hereof, many different types of algorithms may be used to determine, for example, a threshold or decision point during use of system 900.

In another embodiment of an algorithm or methodology, pressure in extending member 910 was sampled with a 24 bit A/D every one second, and averages were generated over a defined period of time. These averages were stored in a ring buffer of the memory system. In a number of representative embodiments, three windowed averages were generated. The three windows were 15, 60, and 15 seconds in size and were end-to-end windows. The outer or 15 second averages were differenced to calculate changes in pressure or deltas (Δs). The deltas were compared against a threshold to determine (in the case of a bed) an “on bed” or “off bed” state or state change. The threshold was calculated via an adaptive algorithm. In that regard, the algorithm stored the maximum delta for each of the last three days. In addition to the three days, a history value was calculated. The history value was calculated/updated by multiplying the current history value by 3 and adding the current day's peak value and dividing by 4. The three daily averages and the history value were averaged together. The threshold was the set to equal to 50% of the resulting average.

HV_{new}=(3*HVₚᵣₑᵥᵢₒᵤₛ+DV)/4

Threshold=0.5*((D1+D2+D3+HV_{new})/4)

wherein HV=history value; DV=daily peak value for the current day; D1=current day peak; D2=yesterday's peak value and D3=peak value from two days ago. The algorithm or methodology is summarized as follows:

a. Generate 1 seconds sample;

b. Calculate average 1, samples s[n]-[n-14];

c. Optionally, calculate average of 60 second window;

d. Calculate average 2, samples s[n-75]-s[n-89];

e. Subtract average 2 from average 1=delta;

f. Compare delta to threshold;

g. Generate ON or OFF event; and

h. Process delta for generating daily max delta.

FIGS. 3H and 3I illustrates results of application of the above algorithm/methodology in two different beds with two different determined adaptive thresholds. As illustrated “ON” and “OFF” events are determine from raw pressure data over time via the above methodology.

As described above, once presence has been determined, system 900 may continue to monitor pressure to look, for example, for relatively small variations in pressure corresponding to movement and/or physiological parameters associated with pressure changes. As described above, the variation in pressure may, for example, be compared to variations in the minimum or baseline pressure. As clear to one skilled in the art, other algorithms to determine movement may be used. If, for example, a pressure of 8 psi is measured and is subsequently relatively constant, the measurement may be associated with the presence of an inanimate object such as a suitcase. If, for example, the pressure varies from 7 to 9.1 psi, the pressure variation may be associated with movement, validating the presence of a person. If a measured pressure associated with presence is maintained, but movement/pressure variation ceases, the cessation of movement may be associated with a problem and an upload upon exception may be initiated.

As describe above, system 900 is adaptive or learns over time. Because no thresholds (for example, associated with presence, movement etc.) are preset or established for system 900 is placed in us, but determined over time and adjusted thereafter on the basis of measured parameters, system 900 may for example, adapt to various changes (for example, a change in weight of person 5). Likewise, system 900 readily adapts to a change in mattress type or cushion type or to being placed in operative connection with a different item (for example, a new bed).

In a number of embodiments, sensor system 900 may also or alternatively be used to monitor clinical items such as heart rate, respiratory rate, movement in general (something in bed, something in bed moving, something in bed within expected limits of a person, physiological parameters) etc. Such items cause measurable pressure and/or flow variation. Sensor system 900 can also be used to trend weight changes over time.

Sensor systems can also be used in connection with one or more medical devices (for example, diagnostic or treatment devices) used in connection with the monitored person's body or medical care. For example, dental CPAP appliances are sometimes used to treat persons suffering from obstructive sleep apnea. Compliance with dental CPAP device therapy is, on average, less than 60% in the United States. One or more sensors can, for example, be used to monitor persons using dental CPAP appliances, and track the hours of usage of such devices. A sensor system can, for example, be placed on the side of the dental CPAP device, which, when in use, resides in the person's mouth and senses the use of the dental CPAP device by, for example, sensing changes in temperature or conductivity in the person's mouth. The data can then be transmitted to remoter system 200 for compliance tracking purposes.

In another embodiment, one or more sensor systems can, for example, be placed in operative connection with a continuous positive airway pressure or CPAP device (or other positive airway pressure of PAP device) often used by persons suffering from obstructive sleep apnea to monitor, for example, compliance. For example, a CPAP sensor can transmit data of the on time, the off time, the usage time, and the average pressure rather than transmitting a stream of analog data, which is then interpreted on the server side.

Persons undergoing treatment for chronic or other health conditions in the home such as obstructive sleep apnea (OSA) and other conditions require frequent monitoring. A comprehensive monitoring program involves the collection of both quantitative and qualitative metrics. While quantitative metrics are most easily collected using sensors and associated devices, qualitative methods generally require an interaction with the person using a variety of systems and/or methods, including conversations over the phone, internet, SMS methods, or via mail.

Using conventional manual methods, a nurse or healthcare provider typically reviews the output of quantitative metrics from sensors and modifies a conversation with person 5 accordingly to collect the most appropriate qualitative data possible. When utilizing automated or semi-automated methods, however, such as IVR, web-based surveys, or similar methods, it is difficult to dynamically change the qualitative data collection based upon sensors, thereby reducing the effectiveness of the qualitative monitor and increasing the number of questions and/or surveys required of person 5 (which contributes to dissatisfaction).

In a number of embodiments hereof, a medical device monitoring device or system (for example, a PAP monitoring device) collects usage, compliance, and clinical efficacy data. The device can be used in conjunction with a management tool incorporated within or operating in conjunction with monitoring system 50 that is, for example, at least partially automated to contact person 5 (utilizing, for example, IVR, SMS, email, and/or internet communication methods) whereby the questions asked and the data collected via the management tool are changed based upon the data being collected from the PAP monitoring device.

For example, current OSA patient management technology asks a patient or person how long and how frequent they have been using their therapy. With the incorporation of the PAP monitoring device, rather than asking how long they've been using their therapy, the management tool can tell them how long they've been using it and offer feedback (positive or negative) to the person. Such a methodology provides a more effective monitoring with higher satisfaction.

As discussed above, transmitting state-based or value-based data (for example, periodically) reduces cost, lowers bandwidth usage, and requires less memory as compared to continuous, real-time transmission of analog data. The transmission of state-based data hereof to remote system 200 may be in a batch manner as described above or may be continuous or substantially continuous in, for example, the case of an available broadband connection between local system 100 and remote system 200. As further described above, in the case of some type of devices such as medical or physiological devices which monitor movement or physiological parameters (for example, temperature, heart rate etc.) it may be desirable to transfer data at very short periods or even continuously. For such monitoring systems it may be desirable to include a communication module in the associated sensor system for continuous transmittal of data to, for example, local data communication device 150 and ultimately to remote system 200. Table 2 provides a summary of several devices describing the functions or activities monitored, the data type to be transmitted to the remote system 200 and whether the transmission of such data may, for example, be periodic or continuous in a number of embodiments hereof.

TABLE 2

| Item being | Description of what | | Periodic and/or continuous |
|---|---|---|---|
| monitored | monitored | Data type | monitoring/uploading |
| Sleeping patterns | Monitor when the person | Hours, Times of changes | Periodic (but may require timed update |
| | is and is not in bed | of status | that is programmable or an hourly |
| | | | update) |
| Television | Monitor when the | Hours, Times of changes | Periodic (daily update may be |
| | television is on and off | of status | sufficient) |
| Refrigerator | Monitor the times that the | Times of changes in | Periodic (daily update may be |
| | refrigerator is opened. | status | sufficient) |
| Oven | Monitor the times that the | Times of changes in | Periodic (daily update may be |
| | oven is on. | status | sufficient) |
| Microwave | Monitor the times that the | Times of changes in | Periodic (daily update may be |
| | microwave oven is on. | status | sufficient) |
| Lights/lamp | Monitor the times that the | Hours, Times of changes | Periodic (daily update may be |
| | light is on. | of status | sufficient) |
| Water | Measure water flow at the | Hours, Times of changes | Periodic (daily update may be |
| consumption | water intake pipe of the | of status | sufficient) |
| | house or at any desired | | |
| | water-using device. | | |
| Patient physiology | Temperature, heart rate, | Depends upon | May be periodic with increased |
| | blood pressure etc. | physiological parameter | frequency of upload or may be |
| | | being monitored | continuous |

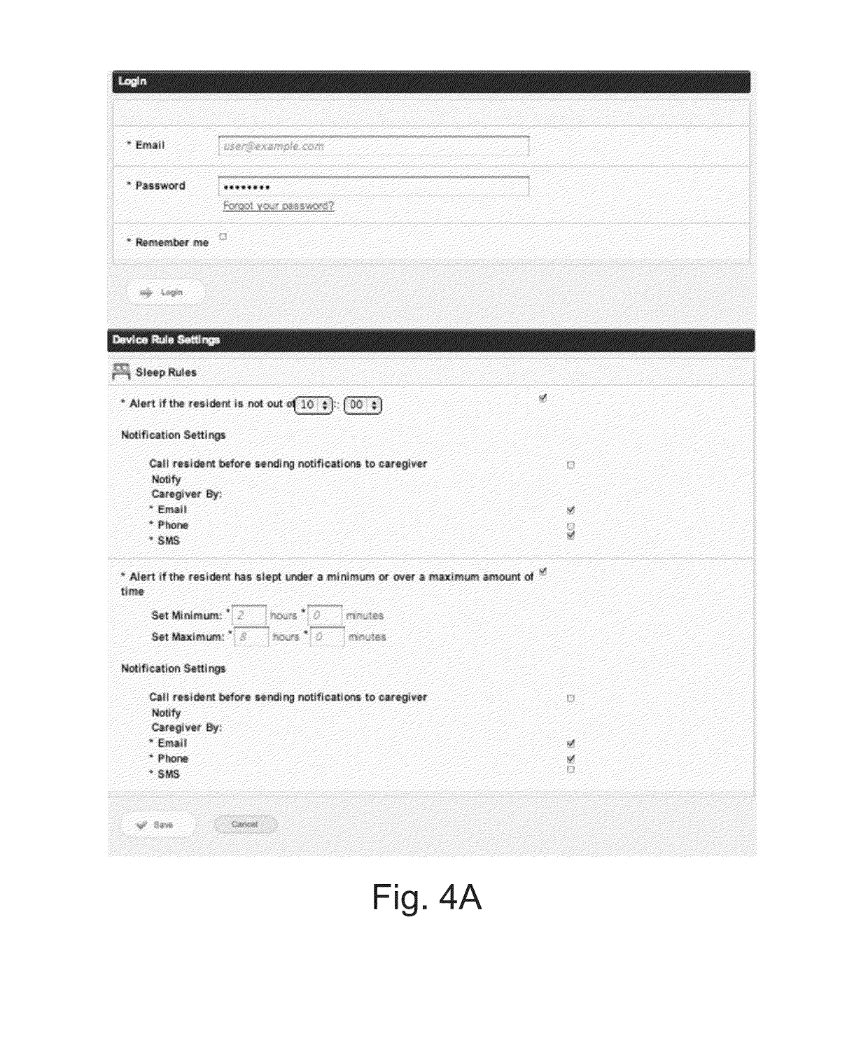

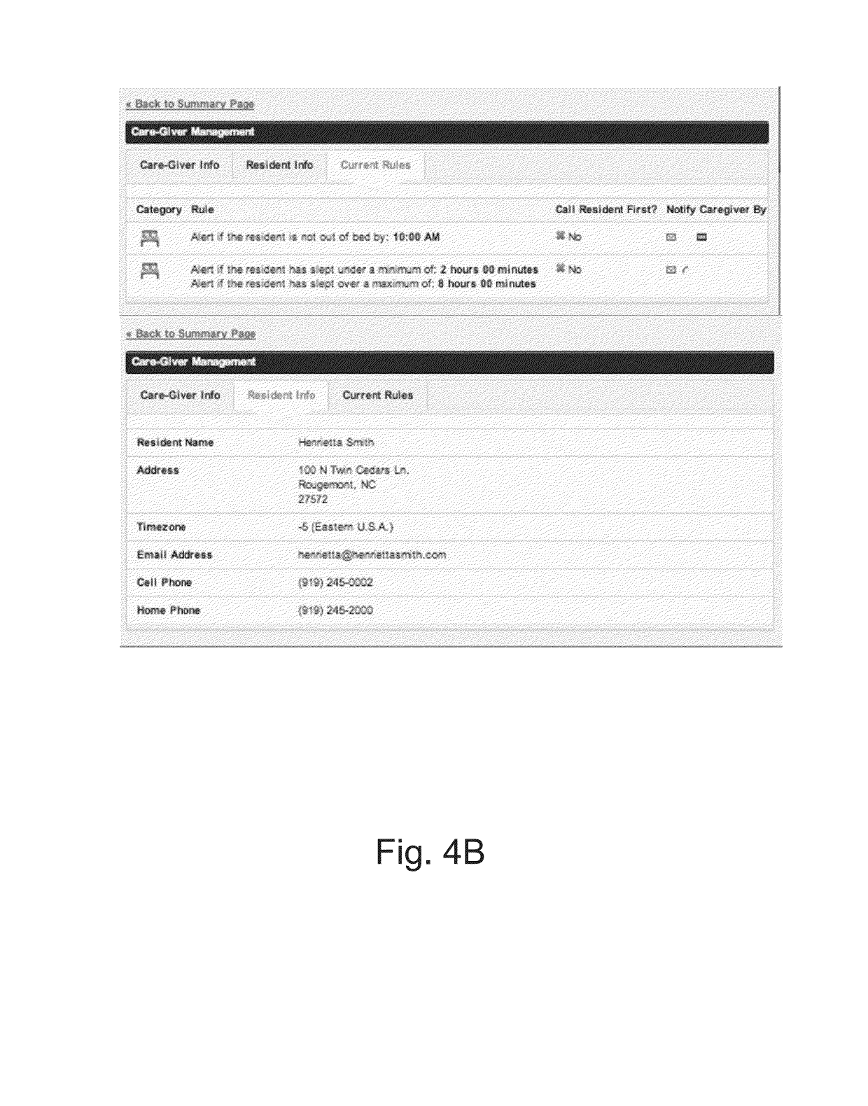

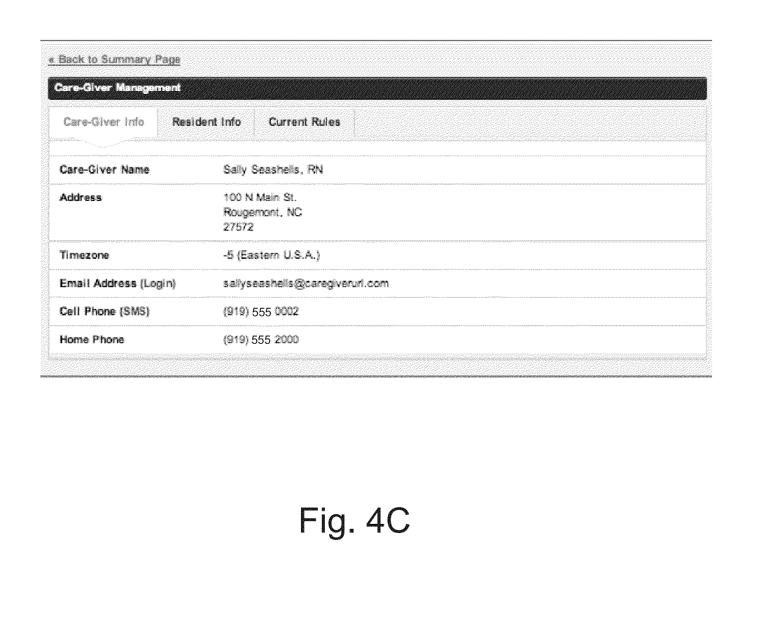

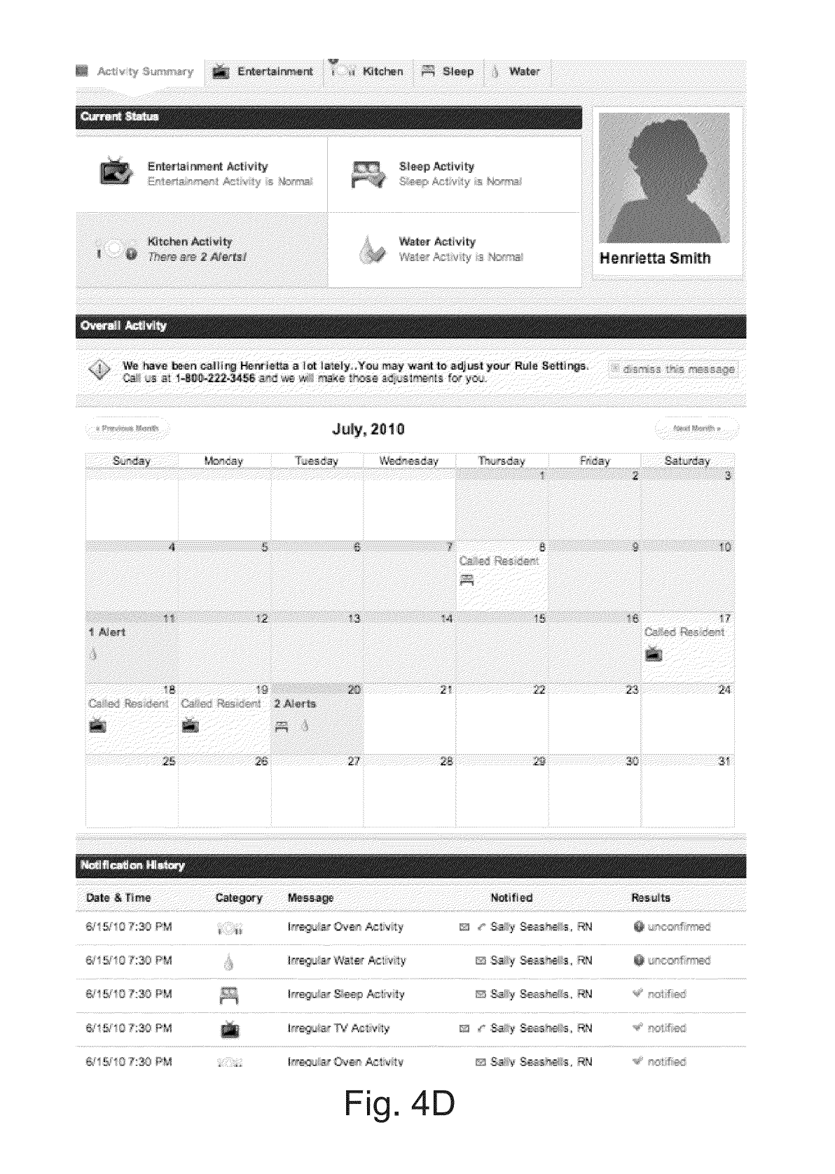

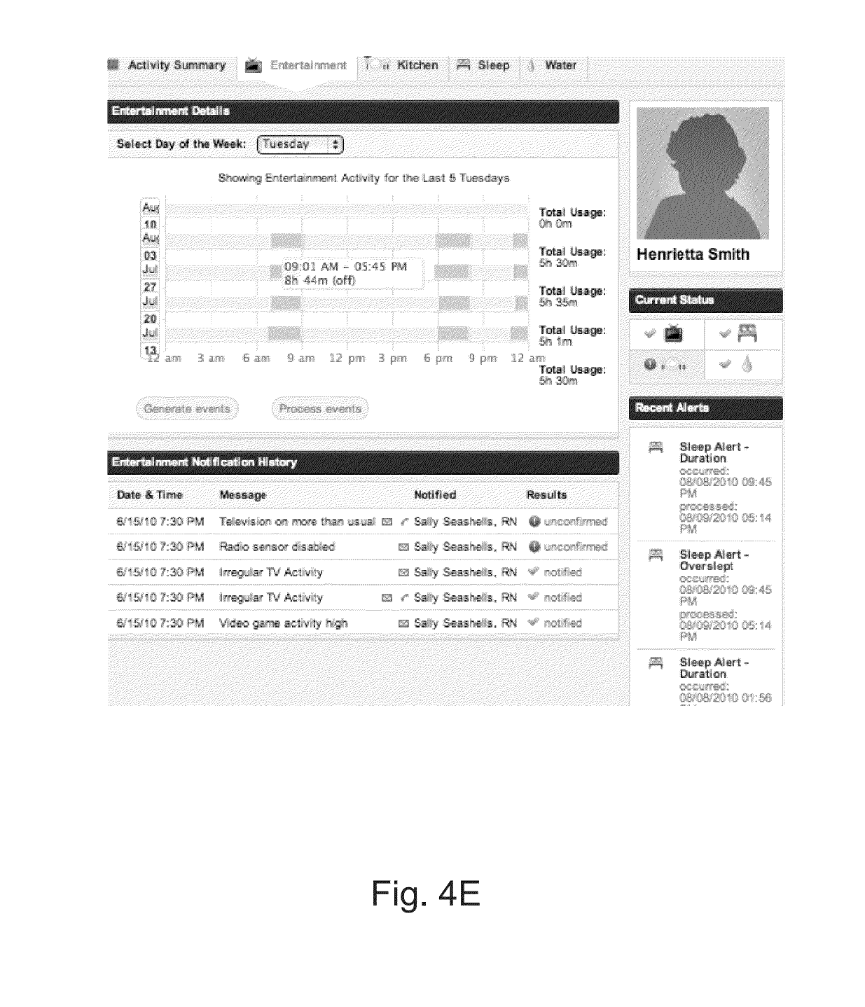

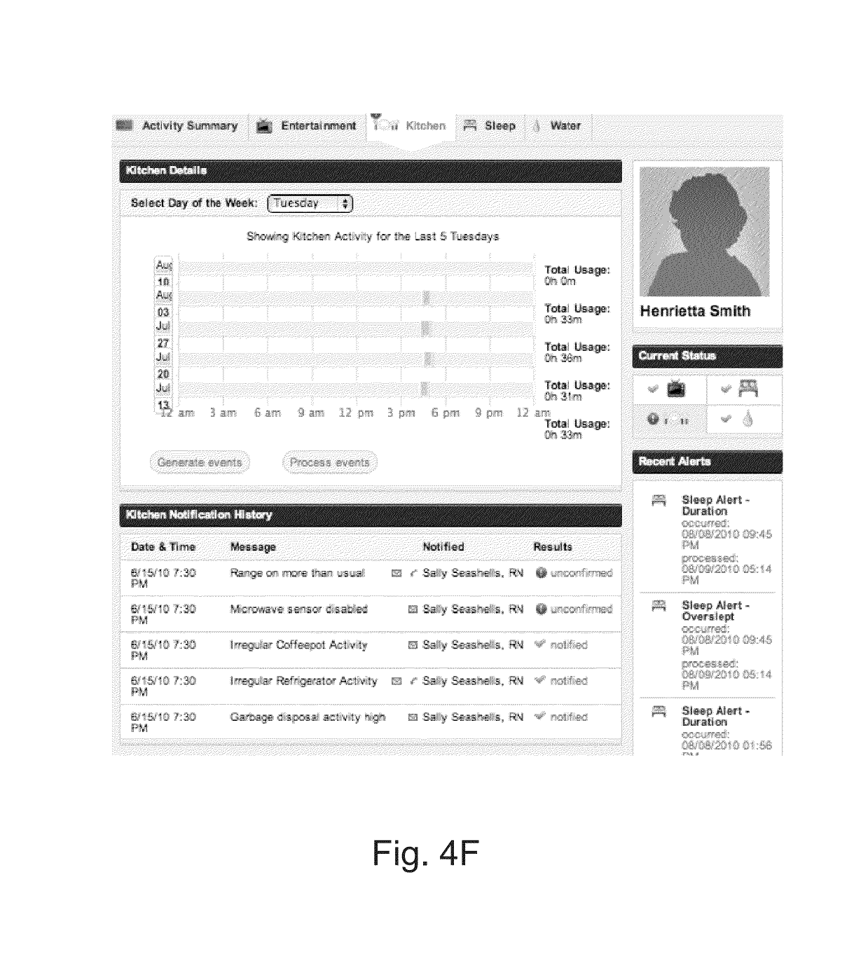

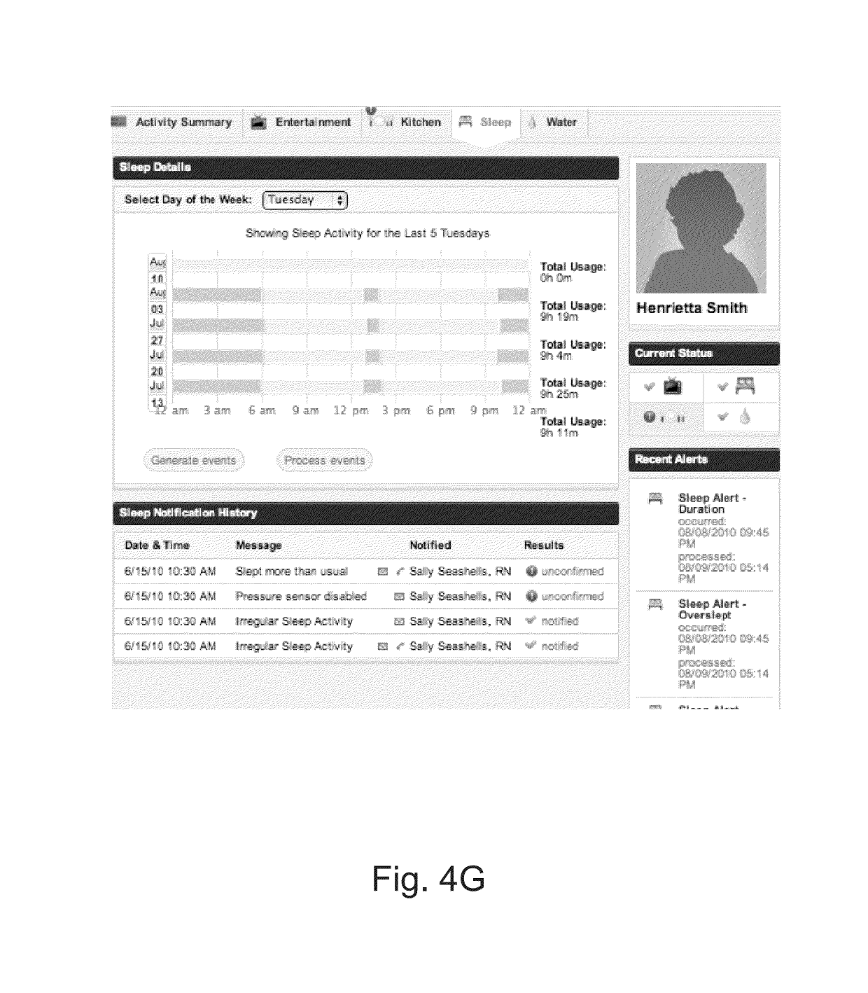

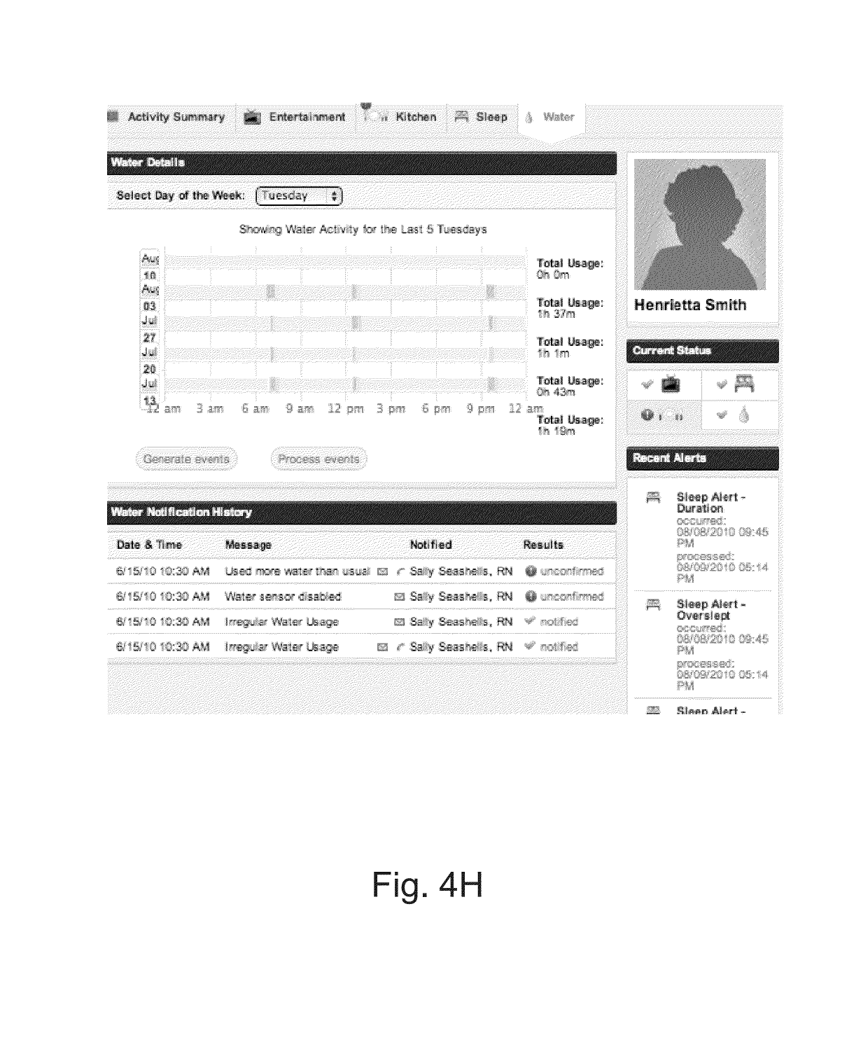

FIGS. 4A through 4H illustrate representative embodiments of computer screen captures from sever-based programming of remote system 200 which are representative of the setup and function of a number of aspects of the systems and methods hereof. In that regard, one or more users or system operators are provided with display/interfaces (for example, web pages via a graphical user interface) to enable setup, configuration, review etc. of monitoring system 50 and the components thereof (see, for example, FIG. 1B).

FIG. 4A illustrates an embodiment of a screen for login and for monitored device rule settings. In that regard, FIG. 4A sets forth a number of rules for the monitored persons sleep activity and associated alerts. FIG. 4B illustrates an embodiment of a screen summarizing rules for alerts to caregivers related to bed activity and an embodiment of a screen summarizing resident information.

FIG. 4C illustrates an embodiment of a screen summarizing caregiver information. FIG. 4D illustrates an embodiment of a screen setting forth an activity summary screen derived from state-based sensor data. Server system 210 can, for example, include logic or learning algorithms to notify an operator of possible modifications (for example, rule changes) that might be desirable to improve operation based upon past actions or experiences (for example, excessive alerts, false alerts etc.) Different categories of activities can, for example, be categorized for ease of viewing and/or analysis. As illustrated in FIG. 4D, a type or category of activity can be selected for viewing and/or analysis from a menu. FIG. 4E illustrates an embodiment of a screen setting forth entertainment activity derived from state-based sensor data from a television, a radio and a computer (video game activity). As illustrated in FIG. 4E, the time of uses and duration of uses can be set forth for a defined period of time. FIG. 4F illustrates an embodiment of a screen setting forth activity derived from state-based kitchen device sensor data from sensor systems associated with a range, microwave, coffeepot, refrigerator and garbage disposal. FIG. 4G illustrates an embodiment of a screen setting forth sleep activity derived from state-based sensor data from one or more sensor systems associated with a bed. FIG. 4H illustrates an embodiment of a screen setting forth water use derived from state-based sensor data from a sensor associated with, for example, a water utility inlet into space 10.

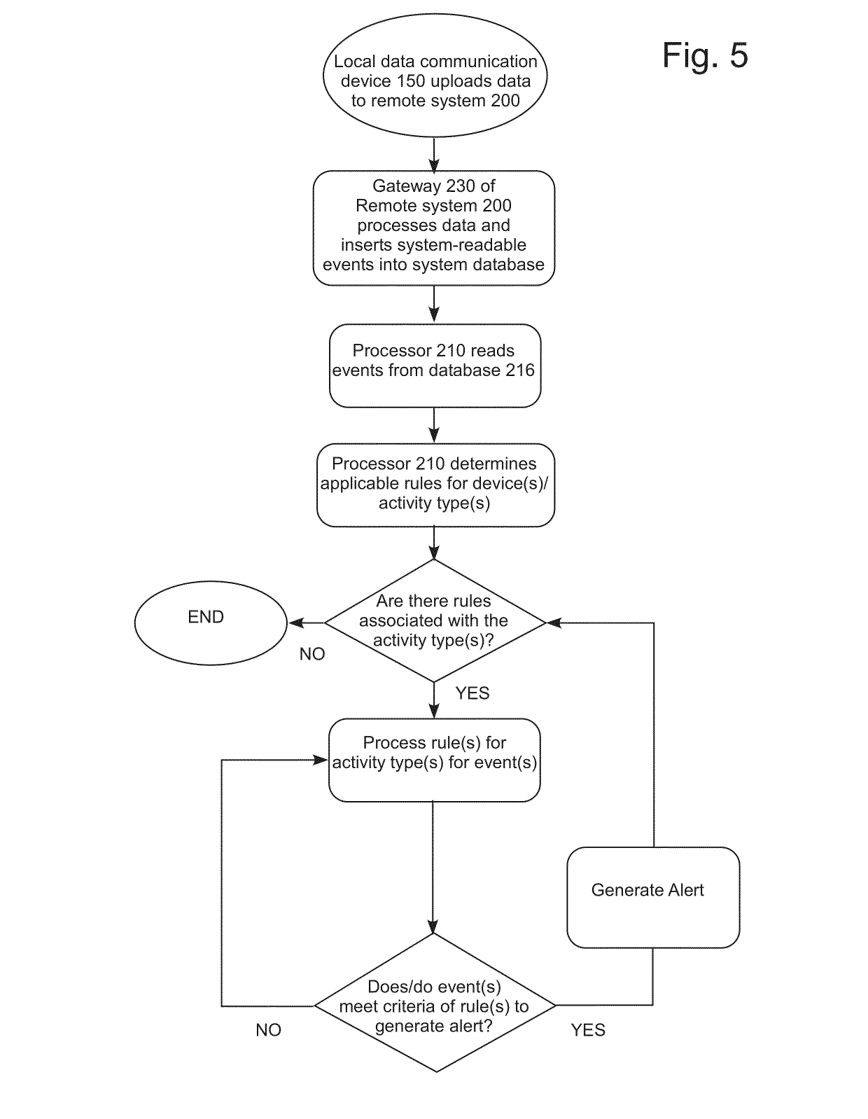

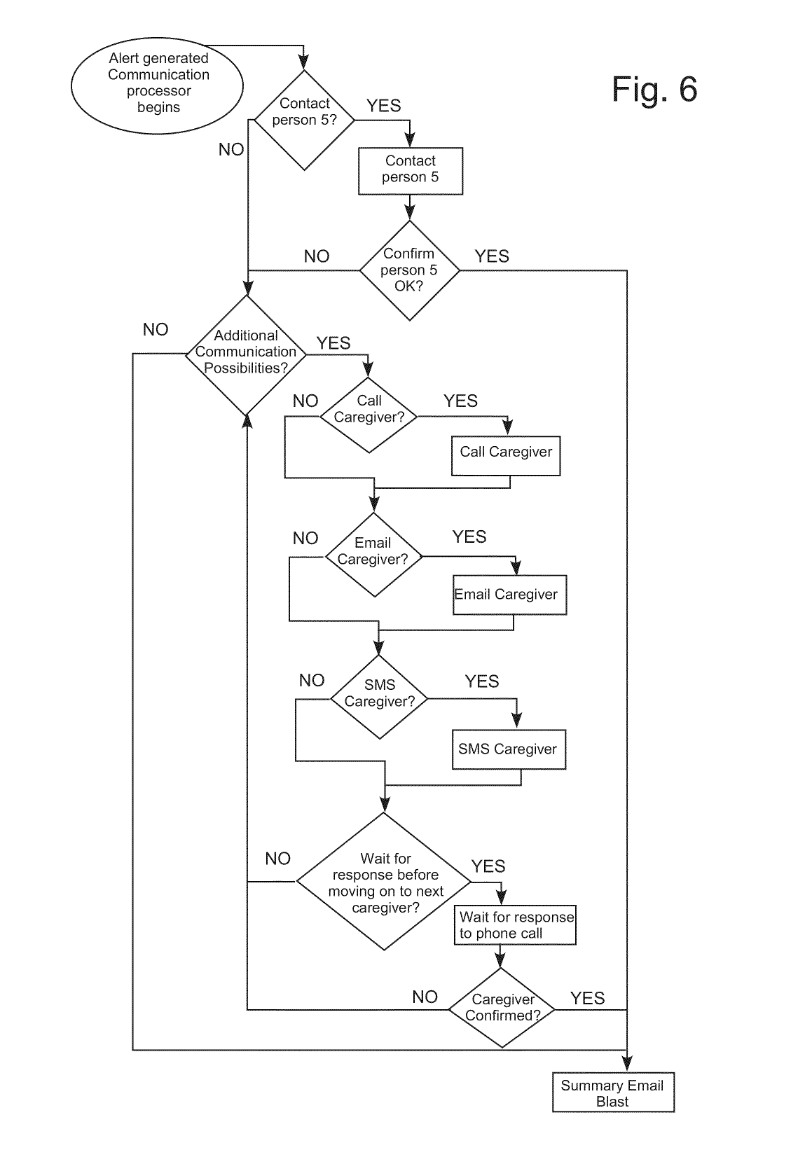

FIG. 5 illustrates a flowchart for an embodiment of methodology for the uploading of data to remote system 200, the determination of associated or relevant rules and the application of such rule to determine whether an alert should be generated. FIG. 6 illustrates a flowchart for an embodiment of methodology for alerting one or more caregivers via one or more communication devices or systems and including an optional attempt to confirm person 5 is OK via an attempt to communicate with or contact person 5.

When monitoring the wellness of person 5, it is necessary to track their behavior on a day to day basis. Such behavior, however, can change at different times of day and from day to day, based upon, for example, whether it is a weekend or a weekday, a holiday or a workday etc. If a wellness monitoring system is designed to generate alerts based upon personal behavior using the same alert thresholds or triggering events at all times/dates, the probability is significant that alerts will be falsely issued or missed on “special” days such as days away from home, weekends, vacations or holidays.

In a number of embodiments, one or more sensitivity settings can be adjusted for specific classifications of time of day and/or dates/days (for example, weekends, holidays, vacations or even seasons of the year). For example, a sensitivity setting can involve a high, medium, or low setting, and corresponding thresholds which change based upon the sensitivity setting and corresponding alerts. Such sensitivity settings result in more accurate alerts (for example, less false positives/negatives.). Moreover the timing of uploads of data from local system 100 to remote system 200 may be altered depending upon time of day and/or dates/days. For example, a frequency of upload may be changed (for example, from three times per day to once per day).

Regardless of system settings, and depending upon personal behavior and monitoring characteristics, there is always the possibility of false alerts being generated. Such false alerts can result in false alarms, lost productivity, and unnecessary expense.

In a number of embodiments of the systems and methods hereof, monitored person 5 can, for example, receive an automatic verification phone call and/or other communication prior to the generation of an alert to one or more remote caregivers. Such a phone call can, for example, attempt to verify that person 5 is in need of assistance to reduce false positives or false alarms.

As described above in connection with uploads upon exception, monitoring various parameters, devices or appliances individually does not take into account information that can be derived by looking at multiple devices at the same time and correlating data therefrom. For example, in the case of a person who has been in bed for a predetermined extended period while the kitchen range is on, in the case that lights are illuminated during off hours for an extended period of time, or in the case that heating/air conditioning settings and/or usage does not correlate with the outside temperature, the person might require assistance. Monitoring of one of these parameters alone or collectively with no correlation of the resultant data may not result in identification of the person's needs. In a number of embodiments, data from sensor systems monitoring devices/systems that are not related or would not be normally grouped together with regard to a particular activity are analyzed to identify anomalies or abnormalities indicative of a condition requiring an action such as an alert or an upload upon exception.

In a number of embodiments of the systems and methods hereof, an array or network of sensor systems operate in concert with each other and data therefrom is correlated such that the wellbeing of the monitored person can be tracked and exceptions and/or alerts can be generated based upon events or values from multiple sensor systems or parameters, tracked in parallel. The data for a plurality (including at least two) sensor systems is thus monitored and correlated using predetermined rules and/or logic to determine if the combination of data from the plurality of sensors indicate the need for an alert. More accurate alerts are thus possible over the case of non-correlated data from individual sensors.

Sensor systems and/or local data communication devices 10 designed to monitor behavior which use a dial up modem, an internet modem or another communication device to transmit data can, for example, be tracked and linked to a specific person based upon a pre-assigned identification code. While such a code identifies the modem or communication device, it does not prevent the device from mistakenly being moved from one location to another. Data transmitted via such a modem or other communication device could be assigned errantly to one person when it actually belongs to another. Because healthcare providers, in the normal course of business, typically move monitoring devices from one person to another, the possibility of errors and errant data transmissions exists.

In a number of embodiments, in addition to the use of a unique identifier associated with a modem or other communication device, the systems and methods hereof incorporate the collection of phone number, IP address etc. from which a modem or other communication device is transmitting data. This information can, for example, be collected in software associated with the device and is linked to an existing person within a database. In the event that a matching phone number, IP address and/or other indication of origin cannot be identified and paired with an existing COM device serial number, the data can, for example, be stored in a staging status until a time when phone number, IP address (for example, a static IP address) etc. can be linked to an existing person. Such identifying data can, for example, reduce errors and reduce or eliminate the potential for errors in data transmission between healthcare providers or caregivers

In addition to wellness monitoring, information from sensor system systems hereof may, for example, also be used for security monitoring of for monitoring for unauthorized use. In that regard, activities sensed by the sensor systems hereof may be associated with an unauthorized access to space 5 or a portion thereof. For example, if space 5 is to be unoccupied for a period of time (for example, during a particular season in the example of the occupant(s) travelling south for winter months), detected activities or a particular type of may be associated with the presence of an intruder. Sensor systems hereof may, for example, be integrated with or placed in communication with many types of security systems in new installations and via retrofitting or addition to existing systems

The foregoing description and accompanying drawings set forth the preferred embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A presence sensor system comprising at least one resilient extending member defining an enclosed sensing volume, the sensing volume comprising a fluid therein, a pressure within the sensing volume changing upon application of force to the extending member, the presence sensor system further comprising a pressure sensor in fluid connection with the sensing volume, a processor system in communicative connection with the pressure sensor and a communication system in communicative connection with the processor system.

2. The sensor system of claim 1 is adapted to determine a pressure threshold associated with onset of presence after being placed in use.

3. The sensor system of claim 2 wherein the system is adapted to change the pressure threshold over time on the basis of measured pressures.

4. The sensor system of claim 3 wherein the pressure threshold is determined on the basis of at least one of minimum pressures measured over time and maximum pressures measured over time.

5. The sensor system of claim 3 wherein the pressure threshold is determined on the basis of at least one of an average minimum pressure determined over time and an average maximum pressure determined over time.

6. The sensor system of claim 2 wherein the sensor system determines a time associated with the onset of presence and a variable associated with duration of presence.

7. The sensor system of claim 6 wherein the sensor system further comprises a flow sensor.

8. The sensor system of claim 6 wherein the sensing volume is filled with air.

9. The sensor system of claim 6 wherein the sensor system is adapted to measure at least one variable associated with the wellbeing of the person after the onset of presence.

10. The sensor system of claim 6 wherein the sensor system is adapted to measure at least one variable associated with the wellbeing of the person after the onset of presence.

11. The sensor system of claim 2 wherein the pressure sensor measures variation in pressure after onset of presence is determined.

12. The sensor system of claim 11 wherein measurement of variations in pressure is used to determine if the onset of presence is associated with a person.

13. The sensor system of claim 12 wherein variations in pressure are associated with at least one of movement or variation in a physiological parameter.

14. The sensor system of claim 13 wherein the physiological parameter is at least one of respiration or pulse.

15. The sensor system of claim 1 comprising a single resilient extending member, wherein the resilient extending member is a tube.

16. The sensor system of claim 1 wherein the sensing volume is filled with air at approximately ambient pressure.

17. The sensor system of claim 3 wherein pressure averages over a defined period of time are determined from measured pressures and a difference between the pressure averages for a subsequent defined period of time and a previous defined period of time are determined.

18. The sensor system of claim 17 wherein the difference is compared to the pressure threshold.

19. The sensor system of claim 2 wherein the output of the pressure sensor is connected to an analog to digital converter in a ratiometric configuration and the analog to digital converter is connected to the processor.

20. The sensor system of claim 1 further comprising an extending support member adjacent to the at least one resilient extending member, the extending support member being more rigid that the at least one resilient extending member.

21. The sensor system of claim 20 wherein the extending support member is adjacent to a side of the at least one resilient extending member generally opposite a side to which force is applied in the case of presence of a load.

22. The sensor system of claim 20 the extending support member is attached to the at least one resilient extending member.

23. A system for monitoring wellness of a person, comprising:
a local system in the vicinity of the person comprising:
a plurality of sensor systems, each of the plurality of sensor systems being adapted to monitor changes in state of at least one monitored system caused by activity or lack of activity of the person, at least one of the plurality of sensor systems being a presence sensor system comprising at least one resilient extending member defining an enclosed sensing volume, the sensing volume comprising a fluid therein, a pressure within the sensing volume changing upon application of force to the extending member, the presence sensor system further comprising a pressure sensor in fluid connection with the sensing volume, a processor system in communicative connection with the pressure sensor and a communication system in communicative connection with the processor system; and
a local data communication device in communicative connection with each of the plurality of sensor system to receive data from each of the plurality of sensor systems.

24. A system for monitoring wellness of a person, comprising: a presence sensor system comprising a resilient extending member defining an enclosed sensing volume, the sensing volume comprising a fluid therein, a pressure within the sensing volume changing upon application of force to the extending member, the presence sensor system further comprising a pressure sensor in fluid connection with the sensing volume, a processor system in communicative connection with the pressure sensor and a communication system in communicative connection with the processor system.

25. A method of monitoring for presence of a person comprising: placing at least one resilient extending member defining an enclosed sensing volume in operative connection with an item upon which presence is to be determined, the sensing volume comprising a fluid therein, a pressure within the sensing volume changing upon application of force to the extending member, measuring pressure via a pressure sensor in fluid connection with the sensing volume, and communicating a signal from the pressure sensor to a processor system in communicative connection with the pressure sensor; and providing a communication system in communicative connection with the processor system.
